(19) United States
(12) Patent Application Publication
(10) Pub. No.: US 2025/0032078 A1
(43) Pub. Date: Jan. 30, 2025

(54) SYSTEMS AND METHODS FOR MANAGEMENT AND TREATMENT OF BONE CONDITIONS

(71) Applicant: Decode Bone LLC, Milpitas, CA (US)

(72) Inventor: Amrish J. Walke, Milpitas, CA (US)

(21) Appl. No.: 18/787,498

(22) Filed: Jul. 29, 2024

Related U.S. Application Data
(60) Provisional application No. 63/516,022, filed on Jul. 27, 2023.

(51) Int. Cl. A61B 6/50; A61K 31/663; A61K 31/675; G16H 20/10; G16H 50/20; G16H 50/30

(52) U.S. Cl. CPC A61B 6/505; A61K 31/663; A61K 31/675; G16H 20/10; G16H 50/20; G16H 50/30

(57) ABSTRACT

The present disclosure includes methods and devices used to diagnose or treat or manage bone conditions such as osteoporosis. The present disclosure includes bone and soft tissue imaging methods, some of which involve specific patient positioning and alignment of bodily regions and ensuring consistent imaging at multiple time points. Several artificial intelligence-based techniques are also disclosed that process imaging data and other clinical inputs using machine learning models and other methods to obtain desired clinical information. The disclosed artificial intelligence-based techniques are designed to use imaging data and other clinical inputs generated by the imaging methods and devices of the disclosure or existing imaging data and other clinical inputs.

### CROSS-REFERENCE TO RELATED APPLICATIONS

This application claims the priority benefit of U.S. Provisional Patent Application Ser. No. 63/516,022, filed Jul. 27, 2023, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of managing bone conditions, and more specifically to the field of managing bone-related conditions such as osteoporosis and osteopenia. Described herein are systems and methods for imaging bone and other tissues and obtaining clinical information related to bones, and other tissues for managing bone conditions.

### BACKGROUND

Osteoporosis is a medical condition characterized by weakened bones, making them fragile and more likely to break. It occurs when the creation of new bone doesn't keep up with the removal of old bone. This imbalance leads to decreased bone mass and density. There are several causes of osteoporosis including aging, hormonal changes, dietary factors, and lifestyle factors. For example, bone density typically peaks in a person's 20s, and decreases over time. Decreased estrogen levels in women during menopause and low testosterone levels in men can accelerate bone loss. Low calcium and vitamin D intake can contribute to weak bones. Lack of physical activity, excessive alcohol consumption, and smoking can increase the risk. Osteoporosis can be treated using a variety of drugs and other therapies, but these therapies can be limited by side effects.

### SUMMARY

There is a need for new and useful system and method for method of obtaining bone-related clinical information. In particular, there is a need for systems, devices, and methods that obtain bone-related clinical information in patients with bone-related conditions such as osteoporosis and osteopenia.

The present disclosure includes bone and soft tissue imaging methods, some of which involve specific patient positioning and alignment of bodily regions and ensuring consistent imaging at multiple time points. The present disclosure includes several methods for obtaining bone-related clinical information using an imaging device and artificial intelligence-based techniques such as machine learning (ML) models. The methods may be used to make treatment decisions such as initiation or non-initiation of a therapy, selecting type of therapy and other parameter(s) such as dose, route of administration, duration, etc. The methods may be used to determine one or more timepoints of subsequent scans and/or follow-ups. Methods of the present disclosure may generate clinical information such as: bone strength, bone mineral density, and other outputs generated by dual-energy X-ray absorptiometry scanners, bone quality, trabecular bone score and other bone scores, fracture resistance, location(s) of bone sites at high risk of fracture, fracture risk, presence or absence of osteoporosis, severity of osteoporosis, predicted or expected progression of osteoporosis, and predicted response to one or more drug or non-drug therapies. Multiple scans may be performed in some embodiments of the present disclosure to track changes in bones and other tissues with time. The disclosure also includes methods for predicting responses to therapies and selection of appropriate therapy(ies) to be administered to a patient. Various imaging devices are included in this disclosure. Such imaging devices may include components such as sources, detectors, radiation limiting mechanisms, sensors, guides, and other tissue positioning mechanisms to image tissue. The imaging devices may contain a computer-readable medium or be in electronic communication with a computer-readable medium. The computer-readable medium may be adapted to store information about the positioning and/or orientation of prior imaging studies. Various AI-based models and other constructs are also disclosed. Several artificial intelligence-based techniques are also disclosed that process imaging data and other clinical inputs using machine learning models and other methods to obtain desired clinical information. The disclosed artificial intelligence-based techniques are designed to use imaging data and other clinical inputs generated by the novel imaging methods and devices of the disclosure or existing imaging data and other clinical inputs.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A shows an embodiment for of a device for imaging one or more portions of an arm.

FIG. 1B shows an embodiment for of a device for imaging one or more portions of a leg.

FIG. 1C shows an embodiment of a device for imaging one or more portions of a femur.

FIG. 1D shows an embodiment of a device for imaging one or more portions of a finger.

FIG. 1E shows an embodiment of a device that comprises a radiation limiting mechanism.

FIGS. 1F-1G show examples of relative movements between system components and tissue regions.

FIG. 1H-1J show examples of gantry-based systems of the present disclosure.

FIGS. 2A and 2B show two X-ray images of human bone (phalanges) before and after a treatment for increasing bone strength.

FIG. 2C shows the changes between FIGS. 2A and 2B calculated using computer-based analysis.

FIGS. 2D and 2E show two X-ray images of human bone (phalanges) before and after a treatment for increasing bone strength.

FIG. 2F shows the changes between FIGS. 2D and 2E calculated using computer-based analysis.

FIG. 2G-2I show the method steps of a method for aligning two or more bone scans.

FIG. 2J-2L show the steps of a method for comparing bone images from two time points.

FIG. 3A shows an embodiment of a method of the present disclosure wherein the target tissue or organ is scanned at least twice.

FIG. 3B shows an embodiment of a method for regularly monitoring the treatment of a patient and optionally adjusting the treatment plan.

FIG. 3C shows an embodiment of a method of generating clinical information using one or more deep learning models.

FIG. 3D shows an example of a flow chart of a supervised ML model that uses labeled data.

FIG. 3E shows a list of selected imaging methods and labels of the present invention.

FIG. 3F shows one such machine learning model being used to predict the treatment outcomes of one or more treatments.

FIGS. 3G and 3H show two method embodiments showing how an AI-based model of the present invention may be incorporated in a treatment method using an imaging system.

FIG. 3I illustrates how multiple input parameters can be considered to determine the treatment plan of a patient.

FIG. 3J shows an embodiment of a method of the present disclosure that includes two therapies wherein a therapy parameter of the first therapy is adjusted.

FIG. 3K shows an embodiment of a method of the present disclosure that includes two therapies wherein the first therapy is administered as a bolus.

FIGS. 3L and 3M show more embodiments of a method of the present disclosure that includes two therapies wherein the first therapy is administered as a bolus.

FIGS. 3N and 3O shows method embodiments of the use of the present disclosure for determining and/or managing a drug holiday.

FIG. 3P show another embodiment of a method of the present disclosure comprising two therapies.

FIG. 3Q shows a method embodiment of sequencing two therapies.

FIG. 3R shows another method embodiment of sequencing two therapies.

FIG. 3S shows another method embodiment of sequencing two therapies.

FIG. 3T shows another method embodiment of the use of the present disclosure for determining and/or managing a drug holiday.

FIG. 3U shows another method embodiment of sequencing two therapies.

FIG. 3V shows another method embodiment of sequencing two therapies.

FIG. 3W shows a method embodiment of sequencing three therapies.

FIG. 3X shows a method embodiment of managing multiple, simultaneous therapies.

FIG. 4A shows a graph of an example change of a bone parameter with time in a female patient through menopause.

FIG. 4B shows an embodiment of the present disclosure wherein an imaging step disclosed herein was performed after menopause.

FIG. 4C shows the use of the present disclosure to determine one or more historical and/or future values of the parameter where a low peak bone parameter is declining slowly.

FIG. 4D shows the use of the present disclosure to determine one or more historical and/or future values of the parameter where a high peak bone parameter is declining rapidly.

FIG. 4E shows the use of the present disclosure to determine one or more historical and/or future values of the parameter where a low peak bone parameter is declining rapidly.

FIG. 4F shows the use of the present disclosure to determine one or more historical and/or future values of the parameter where a high peak bone parameter is declining slowly.

FIG. 4G shows an embodiment of the present invention wherein multiple measurements or one or more bone parameters are used to stop a treatment.

FIG. 4H shows another embodiment of the present invention wherein multiple measurements or one or more bone parameters are used to stop a treatment.

FIG. 4I shows another embodiment of the present invention wherein multiple measurements or one or more bone parameters are used to stop a treatment.

FIG. 5A lists examples of drugs classes, drugs, and their typical delivery routes, and typical dosing frequency.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

There are several bone related diseases where the treating physician does not receive regular feedback on the performance of the therapies being administered. This makes it difficult to regularly titrate the therapies or change therapies. Osteoporosis is one such condition. Despite the availability of several therapies, there are still unmet needs in osteoporosis and/or osteopenia treatment. Many current therapies are limited by their side effects, which are typically correlated to the dose and/or duration of treatment. A technical problem with this approach is that the efficacy of current therapies is often known a few years after starting that drug therapy via a repeat bone mineral density scan. This long gap makes it difficult to regularly titrate the therapies or change therapies. A class of drugs called bisphosphonates is typically the first line of treatment for such patients. They are not efficacious in a large percentage of patients. This lack of efficacy is found only after a few years of taking bisphosphonates. A technical solution to the above technical problem provided herein is an ability to determine earlier whether a drug, for example bisphosphonates, is not showing adequate efficacy in a patient so that the patient can be transitioned to other, more efficacious therapies.

Often, patients may simply stop taking the drugs because of side effects or because they don't see or experience the benefit in taking them. The technical problem with this approach is that it exposes the patient to the serious consequences of fractures such as hip fractures. Fracture prevention is the primary goal of managing osteoporosis and/or osteopenia. The fracture risk of a patient is dependent on several factors such as the bone condition, therapies being administered, lifestyle, etc. A technical solution to the above technical problem, provided by the systems and methods described herein, is to calculate the fracture risk, ideally in the physician's office setting, especially in patients already taking one or more osteoporosis therapies.

In some aspects, the systems and methods described herein provide a technical solution to the above technical problems by enabling frequent or more regular titration of one or more therapies or change therapies of a patient.

In some aspects, the systems and methods described herein provide a technical solution to the above technical problems by enabling receipt of feedback about one or more effects of a therapy.

In some aspects, the systems and methods described herein provide a technical solution to the above technical problems by enabling the consideration of multiple inputs (e.g., therapies, lifestyle, genetic predisposition, bone condition, etc.) to determine a personalized therapy strategy that can be adjusted at regular intervals.

In some aspects, the systems and methods described herein utilize artificial intelligence based techniques, such as machine learning to identify or detect anomalies (e.g., one or more parameters of a bone region or tissue region), such as fractures and/or weakening through both cortices with or without a medial spike; transverse or short oblique fractures and/or weakening orientation; noncomminuted (not reduced to small powder and/or particles) presentation; localized periosteal reaction of lateral cortex; increased cortical thickness of diaphysis; focal cortical change(s); radiolucent line(s); localized periosteal or endosteal thickening of the lateral cortex (“beaking” or “flaring”); asymmetry on comparison with the contralateral femur; microfractures; and early symptoms of atypical bone weakening or fracture, etc. The use of specially trained artificial intelligence based models to detect anomalies realizes a number of improvements over traditional methods of detecting anomalies, including more accurate detection of anomalies in order to incorporate preventative measures in treatment paradigms. The application further provides methods for training artificial intelligence-based models that lead to a more accurate model for detecting anomalies in tissues, bones, etc. For example, the practical application of such artificial intelligence-based models is that artificial intelligence-based models may be used to calculate a fracture risk from impact from various impact angles. In some embodiments, fracture risk from impact may be measured by modeling the strength or impact resistance of the bone along various planes.

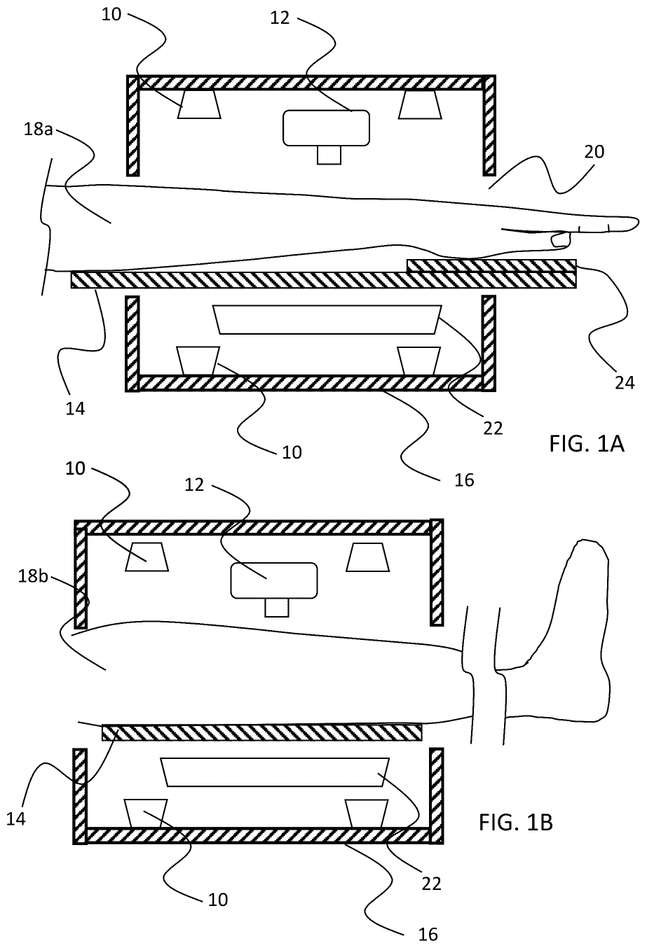

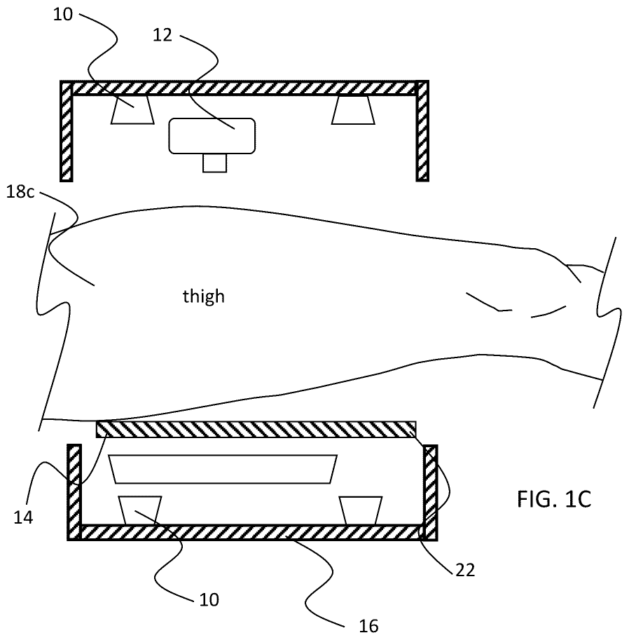

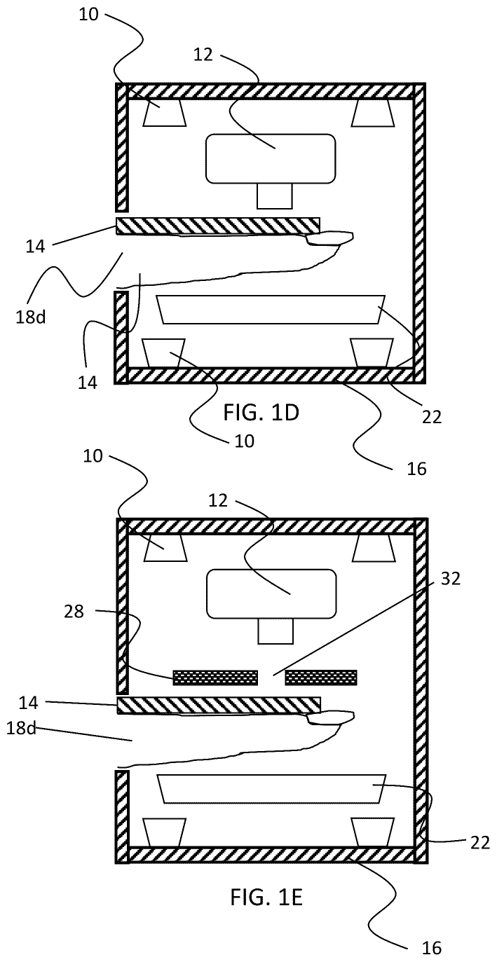

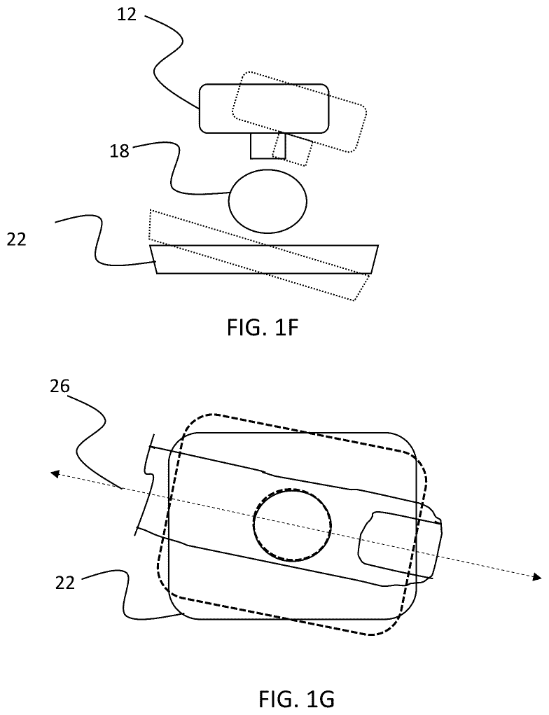

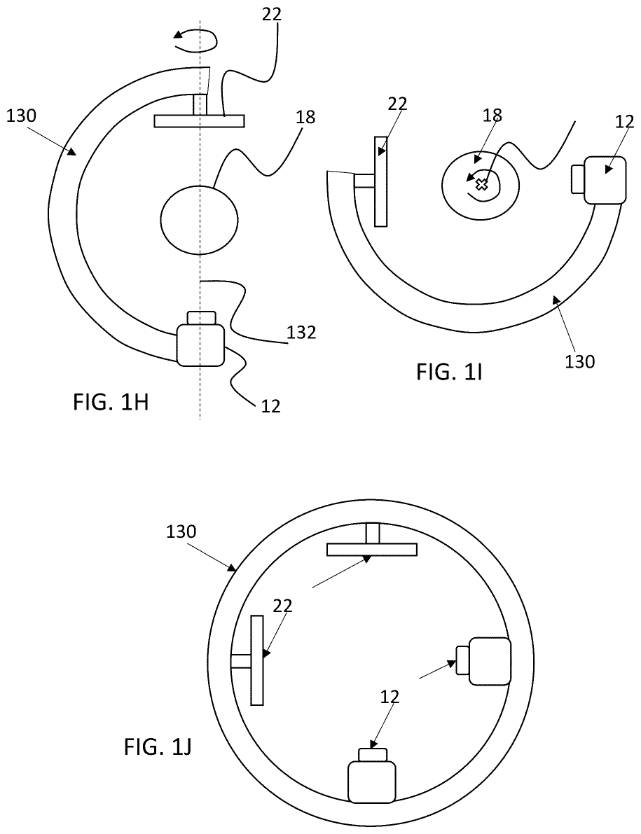

In some aspects, one or more scans or analyses disclosed herein may be performed at one or more sites that are not being treated with a non-drug therapy. Examples of such nondrug therapies include, but are not limited to: vibration therapy (e.g., wearable vibration devices designed for hips, waist, wrists, spine, etc.), organ-specific targeted exercises, etc. Therapies such as vibration-based therapies and organ-specific therapies may be used to increase local bone strength and bone density. Vibration-based therapies may be used to apply gentle mechanical stimulation to regions such as hips and spine to improve bone strength. Examples of vibration-based therapies include, but are not limited to: devices with one or more vibrating elements for imparting repeated mechanical forces to one or more bones at a frequency and acceleration sufficient for therapeutic effect. Such devices may be attached or otherwise placed on a bodily region. FIGS. 1A-1E show various embodiments of the disclosure that demonstrate various system components, relative position of organs/tissues and system component(s), etc. For example, FIGS. 1A-1E show an anatomical region 18 of a lifeform (e.g., human, animal, etc.) at least partially positioned in an enclosure 16. The enclosure 16 may define at least an aperture 20 through which the anatomical region 18 is inserted. The enclosure 16 at least partially houses one or more sensors 10, one or more sources 12 (e.g., x-ray source, radiation source, light source, etc.), and one or more detectors 22. Any of the device embodiments disclosed herein may comprise one or more sensors 10. Several embodiments herein are shown with four sensors 10. However, these are only examples of the number and/or locations of sensors 10 relative to an anatomical region 18. Any of the detectors disclosed herein may be imaging detectors (e.g., photographic plates, X-ray films, etc.), or digitizing devices (e.g., image plates, flat panel detectors, etc.). The enclosure 16 may further include a guide 14 sized and/or shaped to at least partially support, uphold, stabilize, position, or otherwise the anatomical region 18. In some embodiments, the guide 14 may further include a support 24, such as a protrusion, convex region, stepped region, or the like that supports at least a portion of the anatomical region 18. The anatomical region 18 is shown as an arm portion in FIG. 1A, a leg portion (e.g., lower leg, or shin, or tibia, or fibula) in FIG. 1B, an upper leg portion (e.g., thigh, femur, etc.) in FIG. 1C, and a finger portion in FIGS. 1D-1E.

The relative distances and/or orientations between one or more components (e.g., sensor, source, detector, etc.) of an imaging system and one or more tissue region(s) may be matched between two or more scans by any suitable method. In some embodiments, this matching may be performed by visually comparing the scans and repositioning one or more bodily regions and repeating a scan. In some embodiments, one or more anatomical surfaces in an image or on a lifeform (e.g. bone surfaces, skin surfaces, bone images, etc.) may be detected and/or matched between two or more scans using one or more surface fitting methods. Examples of such methods include, but are not limited to:
1. Geometric Fitting: This method involves finding the transformation that substantially or adequately aligns two or more surfaces. This method may include rigid, affine, or non-rigid transformations depending on the complexity of the surfaces.
2. Least Squares Method: In this method, a mathematical algorithm is used to minimize the sum of the squared distances between two or more surfaces. In this method, the best-fit surface is provided by finding the values of parameters that minimize the sum of the squares of the errors.
3. Iterative Closest Point (ICP) Algorithm: In this method, two or more point clouds or surfaces are aligned by iteratively finding the closest points between them and then minimizing the distance between them.
4. Procrustes Analysis: In this method, two or more shapes or surfaces are aligned by scaling, rotating, and translating them to minimize the difference between them.
5. Surface Matching Techniques: These methods use various features of the surfaces, such as curvatures, normals, or edges, to find the best match between two or more surfaces.

To illustrate this, in an embodiment wherein a region of a thigh is imaged using an imaging device, the skin surface of the thigh may be detected. The skin surface data may be stored in a computer-readable medium of the imaging device or in a form that is accessible by the imaging device (e.g., in a computing device communicatively coupled to the imaging device). In a subsequent scan, the skin surface of the thigh may be detected and compared to the stored surface data. If the skin surfaces do not match (e.g. by inconsistent position and/or orientation of the thigh), one or more adjustments (examples of which are disclosed herein) are performed using Geometric Fitting to ensure consistency of the imaging.

The relative distances and/or orientations between one or more components (e.g., sensor 10, source 12, detector 22, etc.) of an imaging device and one or more tissue region(s) may be matched manually by a user by moving (repositioning) one or more regions of the imaging device or tissue region(s). In some embodiments, one or more components of the imaging device and/or tissue region(s) may be moved (repositioned) automatically by the imaging device. Such embodiments of the imaging device may comprise mechanisms such as motors, linear motion mechanisms, etc. to cause the movement.

In some embodiments, one or more skin surface variations or unevenness can be used to align target tissue. For example, for embodiments that image the forearm, the projections of the radius and ulna and the curved space in between them can be used to align the forearm with one or more device components.

In some embodiments, one or more axes (e.g., an axis of a long bone; an axis of a body region such as forearm, upper arm, thigh, finger portions, etc.) may be detected and/or matched between two or more scans using one or more methods.

As shown in FIGS. 1A-1E, a scan orientation and/or distance of tissue from sensor 10 and/or source 12 (e.g., x-ray, radiation, ultrasound, magnetization, light, laser, light-emitting diode, white light, etc.) of a subsequent scan may be matched to that of a previous scan orientation to allow precise comparison of changes across scans. Sensors 10 (e.g., cameras, CMOS/CCD sensors, etc.) may be used to achieve such matching. For example, in some embodiments, the source 12 may be an X-ray source. The cameras, sensors, etc. may be used to determine alignment between one or more tissue regions and portions of the device. To account for changes (e.g., bone changes, skin surface changes, changes in bodily fat distribution, changes in muscle or other soft tissue) with time, systems disclosed herein may recalibrate relevant parameters (e.g., skin surfaces, bone outlines, etc.) with each scan. One or more regions(s) of interest of one or more organs/tissues may be positioned to minimize relative distortion of one or more skin surfaces and/or bones between two scans. As shown in FIGS. 1A-1E, a guide 14 may be used to position one or more organs or tissues to minimize relative distortion of the one or more skin surfaces. A guide 14 may be a planar structure, a structure comprising mechanisms to attach to an anatomical region (e.g. straps, adhesive surfaces, grips, etc.), a structure having one or more concave regions, etc. Minimizing the distortion of skin surfaces and/or bones allows more consistent positioning of the regions(s) of interest between successive scans. For example, minimizing the distortion of skin surfaces allows more consistent positioning of the regions(s) of interest between successive scans in embodiments wherein the skin surface is used to position and/or orient a region of interest. In some embodiments, minimizing distortion is achieved by fixing and/or stabilizing the extremities (e.g., fingers, toes, hand, foot, etc.) of a bodily region (e.g. a forearm, a thigh) and avoiding fixing and/or stabilizing a central portion (e.g., knee, elbow, shin, etc.).

In one or more embodiments herein, multiple orientation markers and/or images may be used. For example, in some embodiments, a combination of a visual skin surface and bone X-ray images may be used. An image of a skin surface and an X-ray image of a bone may be used to minimize distortion by positioning the tissue or organ based on the image of the skin surface and/or the X-ray image of the bone.

One or more surfaces may be detected using techniques similar to “Face ID” wherein a dot projector projects multiple (e.g., >500, >10,000, etc.) dots of visible or infrared light on a surface to illuminate the surface. The illuminated surface may be detected using a camera to create a 3D surface map. One or more surfaces may be detected and/or imaged using any suitable method to generate a mathematical model of the surface. The model may then be checked against a model that was previously set up on the system (e.g., in a previous scan). This checking may be performed using a dedicated neural hardware and machine learning processes that adapt and learn about changes in the one or more surface with time. Examples of methods and devices to detect and/or image one or more surfaces include, but are not limited to:
1. Visual three-dimensional (3D) mapping devices using stereoscopy or photogrammetry. Such device may use stereo cameras that may image while the camera is moved to create a 3D model of the skin.
2. Visual 3D mapping devices using photogrammetry.
3. Devices that detect and/or analyze occlusion of facial features. Such devices may use landmark heat maps to estimate the location of one or more landmarks such as raised surfaces, depressions, etc. on the skin surface. The skin surface may also be mapped through an occlusion heat map. The device may combine multiple such inputs to map the skin surface.

The relative distance and/or orientation of one or more of: sensors 10, imaging systems, emitters, tissues, etc. and one or more of: anatomical region, tissue surfaces, organ regions, etc. may be adjusted to achieve a desired relative distance and/or orientation. This may be achieved by adjusting the position and/or orientation of one or more of: sensors, imaging systems, emitters, tissues, tissue surfaces, organ regions, etc. In some embodiments, a bodily region is fixed to a carrier or platform. The platform may be moved or adjusted (e.g., on sliders, bearings, wheels, etc.) to achieve a desired relative distance and/or orientation between the one or more of: tissue surfaces, organ regions, etc. of the bodily region and one or more of: sensors, imaging systems, emitters, tissues, etc. For example, a guide 14 and/or a support 24 (e.g. as shown in FIGS. 1A-1E) may be movable or adjustable to achieve a desired relative distance and/or orientation. Examples of bodily regions include, but are not limited to: arms, hands, fingers, legs, toes, feet, heads, backs, etc. The relative distance and/or orientation may be adjusted based on the scan parameters of a prior scan such that at least two scans are performed wherein the relative distance and/or orientation is substantially the same between the two or more scans. Any of the adjustments disclosed herein may be performed dynamically i.e., while actively scanning one or more tissue regions using optical scanning, X-ray imaging, etc. FIGS. 1F-1G show examples of relative movements between system components and tissue regions. For example, in FIG. 1G, one or more system components (e.g., detectors 22) may be moved (e.g., translated, rotated) to orient the system component relative to a measured, calculated, and/or detected axis of the organ/tissue, for example an axis 26 of a bone. In this example, a finger is shown as an example of an organ, bone, and/or tissue 18.

In a non-limiting example, one or more fingers (e.g., middle and/or little fingers) may be bent at an approximately 90-degree angle to the plane of a handheld flat to allow insertion into the scanner.

One or more of: sensors, emitters, detectors, imaging systems or components may be fixed on a gantry-based system. The gantry may be designed as a movable platform that supports one or more components of the present disclosure such as sources 12, detectors 122, and sensors 10. Such gantry-based systems may be designed to allow rotational and/or translation motion of one or more system components. The gantry may be translated and/or rotated to perform one or more method steps. FIG. 1H-1J show examples of gantry-based systems of the present disclosure. For example, a gantry-based system may include a detector 122 positioned on a first end of an arm 130, opposite a source positioned on an opposite end of the arm 130. The arm 130 may be rotatable about a rotation axis 132, for example about tissue, organ, or anatomical region 18. FIG. 1J shows a gantry-based system being rotated around an anatomical region 18, wherein the axis of rotation 132 is perpendicular to the axis of anatomical region 18. FIG. 1I shows a gantry-based system being rotated around an anatomical region 18, wherein the axis of rotation 132 is parallel to the axis of anatomical region 18. FIG. 1J shows a gantry-based system 130 having multiple sources 12 and detectors 122.

One or more scans or analyses disclosed herein may be performed at one or more sites that are susceptible to fracture, for example, a femur, hip, wrist, etc. Multiple scans or analyses may be performed on a patient to get information that is used for one or more methods disclosed herein. For example, scans may be performed on one or more hip and/or spine bones in the same patient.

In some embodiments, the scanner may be designed to image trabecular bone with a sufficient image quality (e.g., sufficient resolution) such that the identification and/or analyses of one or more trabecular bone parameters are possible. Examples of such parameters include but are not limited to: trabecular number, trabecular surface area, trabecular separation, trabecular thickness, trabecular bone microarchitecture, mineralization of the trabecular bone, aggregate bone density, bone density distribution, bone outlines, extent, size, shape, uniformity, margins, new bone formation (including new, abnormal formations), new bone loss, bone strength, etc.

Any of the methods and devices disclosed herein may use calibration markers. Examples of such markers include, but are not limited to: wedge markers, spherical markers, cylindrical markers, etc. The outer boundary of markers, e.g., spherical markers, may be used as a marker for distance. In some embodiments, the X-ray density of a middle portion (e.g., 10% of total area) may be used for adjusting the contrast or other parameters in one or more images. In any of the embodiments herein, one or more methods (e.g., use of calibration markers) may be used to match two or more images such that errors due to factors such as differing brightness or contrast are reduced. The location of one or more markers(s) may be determined and/or indicated by the system, for example, by a laser that illuminates the desired marker position. In some embodiments, an initial scan is used to fix the position of one or more markers.

FIG. 1A shows an embodiment of a device for imaging one or more portions of an anatomical region 18 (e.g., the forearm 18a). FIG. 1A shown an arm that is supported or guided by a guide 14. The patient may rest one or more bodily portions on such guides shown herein. FIG. 1A further shows a support 24 that is used to achieve consistent positioning across scans. Examples of supports 24 include, but are not limited to: grips, handles, pads, curved regions for accepting a bodily region, etc. The guides 14 and/or supports 24 may immobilize the anatomical regions 18 such that the scanned tissue doesn't move significantly during imaging. One or more portions of the guides and/or supports may act as radiation shield 28, as shown in FIG. 1E. One or more portions of the guides and/or supports may themselves be or may contain an attached X ray absorption marker (e.g., metal wedge, zones of known X-ray attenuation parameters, etc.) for visualization using X-ray.

One or more embodiments herein may be used to image tissue and bones. Examples of such bones include, but are not limited to: axial skeleton bones (hip, spine, dental, ribs, etc.) or peripheral skeleton bones (arms, hands, fingers, legs, foot, neck, skull, teeth). Suitable systems may be designed that can image the bodily region containing the target bone. Several embodiments in this disclosure may be used for imaging long bones (e.g., bones of arms or legs) of the peripheral skeleton.

FIG. 1B shows an embodiment of a device for imaging one or more portions of a leg 18b.

FIG. 1C shows an embodiment of a device for imaging one or more portions of a femur 18c. Such embodiments may be used for detecting the early onset of and/or increased risk of Atypical Femur Fracture (AFF). For example, as shown in FIG. 1C, a region of femur 18c between lesser trochanter and supracondylar flare may be scanned. The scanning and/or analysis may be performed to detect and/or measure one or more of: fracture and/or weakening through both cortices with or without a medial spike; transverse or short oblique fractures and/or weakening orientation; noncomminuted (not reduced to small powder and/or particles) presentation; localized periosteal reaction of lateral cortex; increased cortical thickness of diaphysis; focal cortical change(s); radiolucent line(s); localized periosteal or endosteal thickening of the lateral cortex (“beaking” or “flaring”); asymmetry on comparison with the contralateral femur; microfractures; and early symptoms of atypical bone weakening or fracture, etc.

In some embodiments, a baseline scan may be performed at or around the time of starting a bisphosphonate therapy. One or more bone parameter(s) may be monitored using the present disclosure during the bisphosphonate therapy. One or more changes in bone parameters during the bisphosphonate therapy may be monitored and/or analyzed at one or more times. In some embodiments, the bisphosphonate therapy may be administered for about 3 years to about 7 years. Bone parameters include, but are not limited to any of the parameters disclosed herein. Bone parameters may be used to monitor safety (e.g., AFF risk) and/or efficacy (e.g., increase in BMD, increase in bone strength, etc.). If patient's risk of AFF is high or adequate efficacy is not determined, the bisphosphonate therapy may be stopped, and the patient may be started on Denosumab or osteo anabolics like Romosozumab.

In some embodiments, a baseline scan is performed at or around the time of starting an anti-resorptive therapy e.g., bisphosphonate or denosumab therapy. One or more bone parameter(s) may be monitored using any of the methods and devices described herein during the bisphosphonate therapy. The risk of atypical femoral fracture or osteonecrosis of the jaw (ONJ) is monitored using one or more embodiments of described herein. If the imaging show sufficiently increased risk, the anti-resorptive therapy may be stopped or paused (drug holiday). Osteoclasts may be allowed to break down sufficient bone such that the risk of AFF is reduced. Thereafter, the anti-resorptive therapy may be restarted.

FIG. 1D shows an embodiment of a device for imaging one or more portions of a finger 18d (e.g., one or more metacarpals or phalanges). The general design of the embodiment of FIG. 1D is similar to the embodiment shown in FIG. 1C. The embodiment in FIG. 1D includes a guide 14 and/or one or more supports 24 to support and/or immobilize one or more regions of a finger 18d such that the finger doesn't move significantly during imaging and can be consistently positioned and/or oriented between imaging studies. Guides 14 and/or supports 24 disclosed herein support and/or immobilize and/or orient one or more anatomical regions during imaging. This ensures that the anatomical region is consistently positioned and/or oriented across imaging studies. Thus, even if the imaging study is repeated at a later time point (examples of such embodiments are disclosed herein) the anatomical region is consistently positioned and/or oriented. Another advantage is that even if the imaging study is repeated using a different imaging device, the anatomical region is consistently positioned and/or oriented.

FIG. 1E shows an embodiment of a device that includes a radiation limiting mechanism. One or more radiation limiting mechanisms may be used to limit the radiation exposure to the patient or to a user. In this embodiment, the radiation limiting mechanism is a shield 28 defining one or more apertures 32 through which radiation may pass. The position and/or orientation of such shields 28 or apertures 32 may be automatically adjusted by the device. Other examples of radiation limiting mechanisms include, but are not limited to: sufficiently thick walls or enclosures that absorb radiation, curtains or other movable barriers that cover one or more openings in the devices, windows including a spring-powered sliding shield that closes the insertion point of an organ, preventing the system for starting until it detects a tissue inside the unit, flaps or other floppy or hard units that close an opening when not in use, user prompts to start, for example when a body part has been inserted into the device, etc. Such radiation limiting mechanisms may be present on any of the device embodiments disclosed herein.

In some embodiments, the distance of the imaged tissue (e.g., skin surface of the imaged tissue) from an X-ray source and/or the detector is less than about 5 cm.

Any of the systems and methods may use auto contrast or other features to automatically adjust the scanning output.

X-ray dose or intensity used in any of the systems and methods herein may be vary at different regions. During imaging, one or more regions may be masked or otherwise filtered from the radiation. This may be done, for example, to expose the region of interest to radiation or to give different doses to regions with different absorption characteristics.

Any of the imaging methods disclosed herein may be performed on bone such that both the trabecular structure and cortical structure may be imaged together. Any of the imaging methods disclosed herein may analyze one or more bone images for fracture propagation sites. Such information may be used for determining fracture risk. Any of the imaging methods disclosed herein may analyze one or more bone images for crack propagation characteristics.

Various portions of the imaged tissue may be color coded according to their specific composition when displayed and/or stored. In some embodiments, the composition may be determined by the X-ray absorption characteristics of the region. In some embodiments, the difference in X-ray absorption between high energy and low energy X-rays is used to determine the specific composition of the tissue regions.

The systems and methods disclosed herein may use X-ray magnification wherein a sensor captures a magnified image because of sensor, tissue, and/or source placement. Any of the X-ray devices disclosed herein may generate X-rays in a cone-beam geometry. In such devices, the X-ray beam divergence may be used to magnify the tissue image. The magnification factor may be fixed or adjustable. The magnification factor may be adjusted by adjusting the ratio of SOD (source-to-object distance) and SDD (source-to-detector distance). In order to achieve high resolution, a higher SDD and/or lower SOD may be used. In some embodiments, the SOD may be less than about 5 cm. In some embodiments, the SDD may be more than about 5 cm.

Any of the devices disclosed herein may use X-ray microscopy to produce magnified images of the target tissues. Such devices may use the difference in absorption of soft X-rays in the water window region (about 280 eV to about 530 eV) by carbon and oxygen atoms. Such devices may use the difference in absorption of calcium atoms and carbon and/or oxygen atoms. Microfocus X-ray designs may be used to achieve high magnification by projection. A microfocus X-ray source may produce X-rays from a small focal spot (e.g., less than about 100 μm). Larger focal spots (e.g., up to about 1 mm) may also be used.

In some embodiments, an initial scanning may be performed using a low X ray dose. The orientation of the target tissue may be determined based on one or more images obtained using the low X ray dose. Thereafter, multi-axis adjustments (e.g., by robotic arm, tilting attachments, etc.) may be used to adjust the relative orientations of the source, sensor, and/or anatomical region. Subsequent scanning may be performed using higher an X-ray dose in a desired orientation.

In some aspects a technical difference of the disclosed systems and methods described herein is that radiated hard tissue as well as soft tissue may be imaged or analyzed. Imaging soft tissue may be used for one or more of: detecting malignancy or malignant or pre-malignant changes from radiation exposure, measuring bone marrow health, measuring bone marrow edema (this can be from recent fracture), measuring tissue health parameters like perfusion, measuring changes in one or more muscles, measuring changes in fat, measuring changes in vasculature or blood supply, etc.

The scan parameters (e.g., resolution, accuracy, orientation, etc.) between two or more scans may be sufficient to determine effects or differences between the two or more scans disclosed herein. Examples of such effects or differences include, but are not limited to: tapering of treatment effect, lack of early therapeutic effect, increase in therapeutic effect, reduction in therapeutic effect, etc.

In some embodiments of the disclosure, the scanner may be designed to image cortical bone with a sufficient image quality (e.g., sufficient resolution) such that the identification and/or analyses of one or more cortical bone parameters are possible. Examples of such parameters include but are not limited to: bone microarchitecture, mineralization of the bone, bone density, outlines, extent, size, shape, uniformity, margins, new bone growth, new bone loss, bone strength, bone turnover rate, etc.

Any of the analyses disclosed herein may be performed using scanning videos. For example, after the position and/or orientation of scanner and tissue may be fixed, the scan may be performed in video mode around an axis. In such embodiments, the imaging may be performed in a cine mode wherein a series of rapidly recorded multiple images are taken at sequential cycles of time. They may be displayed or saved or analyzed in a dynamic movie display format.

One or more image transformations may be used to transform one or more images of the present disclosure. The transformation operation may be performed during one or more steps including, but not limited to: preprocessing data, augmenting data during training, and post processing data.

Examples of such transforms include, but are not limited to the following transforms in MONAI (Medical Open Network for AI):
1. LoadImage: Load medical specific formats file from provided path
2. Spacing: Resample input image into the specified pixdim (Pixel dimensions and units function)
3. Orientation: Change the image's orientation into the specified axcodes
4. RandGaussianNoise: Perturb image intensities by adding statistical noises
5. NormalizeIntensity: Intensity Normalization based on mean and standard deviation
6. Affine: Transform image based on the affine parameters
7. Rand2DElastic: Random elastic deformation and affine in 2D
8. Rand3DElastic: Random elastic deformation and affine in 3D

The systems and methods disclosed herein may be used to calculate the fracture risk from impact from various impact angles. In some embodiments, fracture risk from impact may be measured by modeling the strength or impact resistance of the bone along various planes.

One or more scans or other methods and devices disclosed herein may be used to determine bone parameters of one or more bones and/or changes in bone parameters of one or more bones. Examples of such bone parameters include, but are not limited to:
1. Abnormal deposition in patients taking drugs like anti-resorptives
2. Average density. Examples of this include, but are not limited to: BMD (areal, volumetric, etc.), t-score across an organ or an ROI, z-score across an organ or an ROI, etc.
3. New deposits such as bone mineral deposits
4. New losses such as bone mineral losses. These losses may be determined across multiple regions. Especially detectable losses in a short (e.g. <1 year) period of time indicating rapid progression.
5. Density distribution
6. Effect of drugs in building bone. E.g., by comparing the changes during treatment.
7. Physical side effects of drugs. If significant side effects are found, a therapy may be stopped or modified. Other therapies may be started.
8. Increased or decreased radiolucency at one or more regions
9. Cortical measurements: e.g., thickening or thinning, percentage of bone thickness, boundary or margin characteristics. Boundary and/or margin characteristics may be used as a guide to calculate bone and/or mineral loss. Examples of boundary and/or margin characteristics include, but are not limited to: outline, thickness and/or location, prominent deposits and/or losses, pitting, unevenness, etc.
10. Trabecular pattern(s)
11. Trabecular bone score(s) (TBS)
12. Alterations to trabecular pattern. E.g., altered trabecular micro architecture.
13. Bone porosity
14. Presence of fracture(s) including microfracture(s) and/or microcrack(s) or macro crack(s).
15. Risk of fracture
16. Cracking and/or fracturing characteristics. Examples of which include, but are not limited to: bone strength, flaws, crack nucleation and/or initiation sites, crack propagation sites, crack propagation risk, fracture toughness, stress concentration sites, fatigue resistance, etc.
17. Identifying nuclei of new bone material deposition. Tracking the change in those nuclei over time.
18. Structure, size, and/or other parameters of blood vessels
19. Soft tissue measurement parameters
20. Digital biomarkers of one or more bone or soft tissue regions. Such digital biomarkers may be used as companion diagnostics for one or more drug or non-drug therapies.

In any of the embodiments herein, bone strength (structural strength of bone) may be calculated by one or more methods including, but not limited to: mineralization of the bone, finite element analysis based on inputs from a scan, bone quality analysis, etc. In any of the embodiments disclosed herein, bone strength may be calculated or defined in terms of resistance to an impact.

In some embodiments, a scan parameter may be correlated or otherwise used to determine a bone strength parameter. In one such embodiment, an artificial intelligence-based method such as a machine learning algorithm may be trained using bone scan datasets (including, but not limited to the examples disclosed herein) and also additional datasets (e.g., computed tomography datasets, quantitative computed tomography (QCT) datasets, etc.). One or more bone strength parameters may be extracted from the additional datasets (e.g., bone strength calculated from finite element analysis (FEA)). FEA may be performed to simulated or measure bone strength based on axial compression, side loading, etc. For gathering training or other data, clinical computer tomography (QCT) may be used on human bones to collect FEA data that may be utilized in one or more embodiments disclosed herein.

Any of the X-ray systems disclosed herein may be a DXA (dual-energy X-ray absorptiometry) system. One or more datasets generated by a DXA scanner (e.g., bone mineral scans, soft tissue scans, total tissue scans, single energy scans, femoral scans, hip scans, spine scans, etc.) may be used singly or in combination with other imaging datasets to perform any of the embodiments disclosed herein.

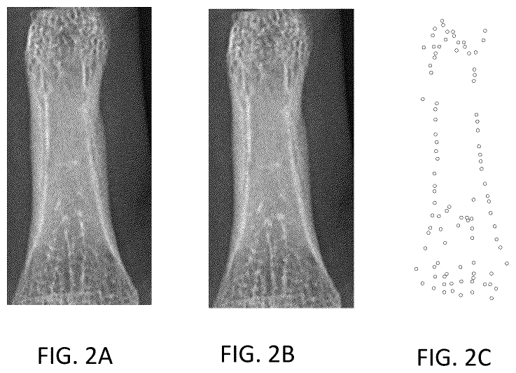

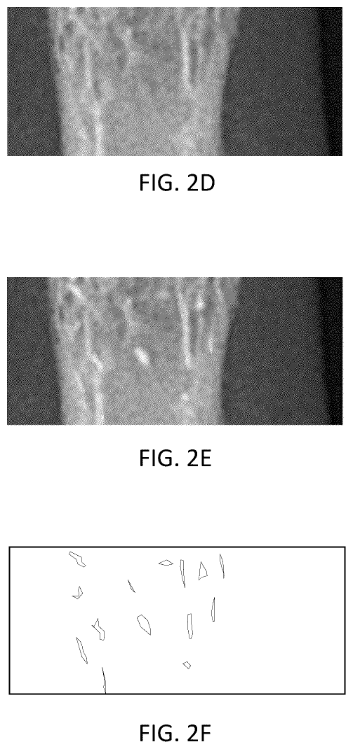

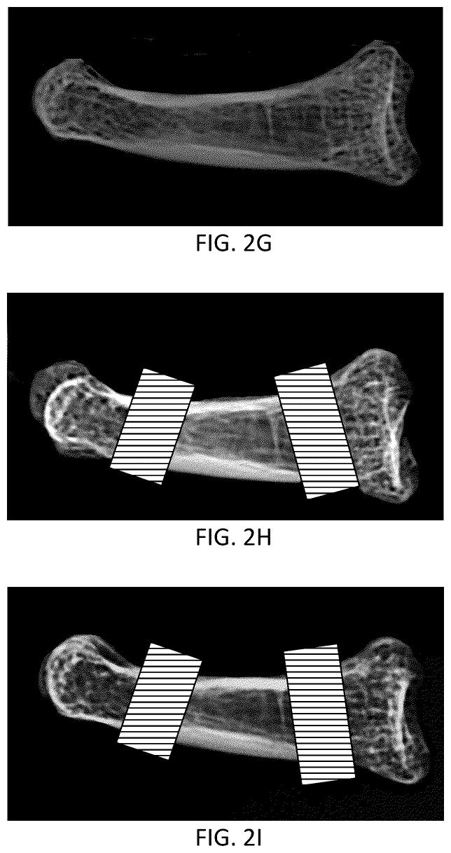

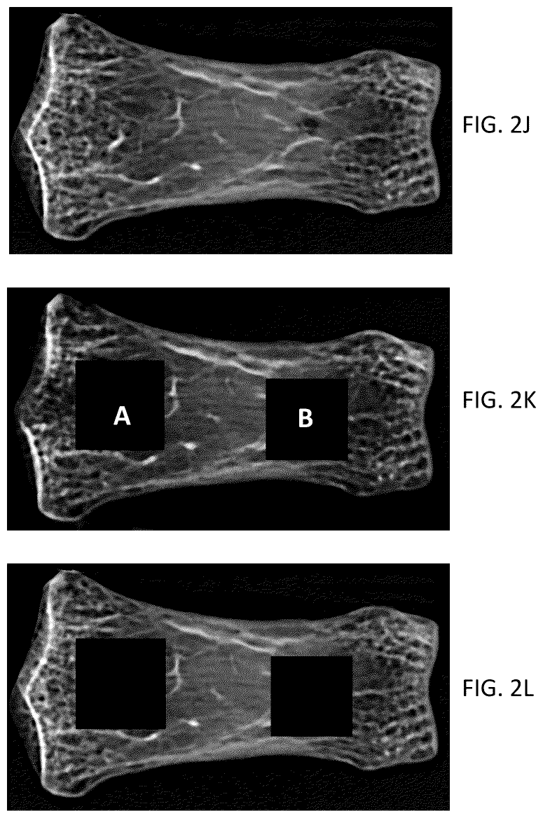

FIGS. 2A and 2B show two X-ray images of human bone (phalanges) before and after a treatment for increasing bone strength. As shown, the orientation and/or position of the bone has been matched between both scans. FIG. 2C shows the changes between FIGS. 2A and 2B calculated using computer-based analysis. FIG. 2C shows multiple points of enhancement in the bone including, but not limited to inner boundaries of cortical bone, upper trabecular region and lower trabecular region. Such calculations at one or more bone or soft tissue regions may be used in the methods disclosed herein. Examples of calculations include, but are not limited to: calculating the bone growth, calculating bone loss, calculating soft tissue growth, calculating soft tissue loss, calculating one or more artifacts, subtracting and/or masking the regions of artifacts, etc. Such enhancements can be used for one or more purposes including, but not limited to: determining amount of newly formed bone, determining rate of bone growth, determining tapering of treatment effect, determining bone loss, deciding to stop a current therapy and start another therapy, determining an individualized therapy for the patient, etc. The multiple points of enhancement in the bone may be displayed and/or analyzed as pixels or other small markers as shown in FIG. 2C.

FIGS. 2D and 2E show two X-ray images of human bone (phalanges) before and after a treatment for increasing bone strength. As shown, the orientation and/or position of the bone has been matched between both scans. FIG. 2F shows the changes between FIGS. 2D and 2E calculated using computer-based analysis. FIG. 2F shows multiple zones of enhancement in the bone including, but not limited to, inner boundaries of cortical bone, inner trabecular region, etc. The multiple zones may be displayed as shown in FIG. 2C and used for one or more purposes described herein.

FIGS. 2G-2I show the method steps of a method for aligning two or more bone scans. Such alignment may be done in any of the methods and devices disclosed herein to allow an accurate comparison of bone scans from the same bone at two different time points. FIGS. 2H-2I show masked areas for hiding one or more artifacts that were introduced in the experiments to cause bone loss. FIG. 2G shows a first (e.g., baseline) scan of a region of bone. In this example, a phalangeal bone is used to demonstrate the method steps. FIG. 2H shows a first scan of the same bone at a subsequent time point. The alignment of the bone seen in FIG. 2H is then compared to the alignment of the bone seen in FIG. 2G. Since they are not well aligned, one or more steps disclosed herein may be used to obtain a subsequent scan (shown in FIG. 2I) wherein the bone alignment is sufficiently similar to that in FIG. 2G. This substantial matching of alignment allows an accurate comparison of the changes in the bone and soft tissue between scans. This substantial matching of alignment may be performed by one or more of: moving the tissue and moving one or more system components. The alignment may be changed while continuously imaging the bone. One or more parameters may be measured in the subsequent scan including, but not limited to: outer boundary of bone, size of bone, shape of bone, alignment of the bone axis, etc. Such measured parameters may be used to change the alignment in the subsequent scan(s) to match bone alignment with a previous scan.

The alignment between two or more scans of the same tissue taken at two or more different time points may be checked and modified using one or more parameters for accurate alignment. Such parameters include, but are not limited to:
1. Orientation relative to an axis of the bone. In some embodiments, the alignment is changed such that the difference in orientations between two scans is less than about 5% (e.g., less than about 2%) in terms of one or more of: roll, pitch, or yaw.
2. Pixel superposition. In some embodiments, the alignment is changed such that the pixel superposition between two scans is better than about 95% (e.g., better than about 99%).
3. Size match. In some embodiments, the alignment is changed such that the size match between two scans is better than about 95% (e.g., better than about 99%).
4. Shape match. In some embodiments, the alignment is changed such that the shape match between two scans is better than about 95% (e.g., better than about 99%).
5. Overlap of key features. In some embodiments, the alignment is changed such that the overlap of key features between two scans is greater than about 95% (e.g., better than about 99%).

As shown in FIGS. 2G-2I, the same bone shown in FIG. 2G was subjected to a loss of bone minerals as seen in FIG. 2H. This example is being used to demonstrate the progression of osteoporosis or other diseases causing progressive bone loss. Such regular monitoring of bone loss may be used to determine clinical decisions including, but not limited to: determining an individualized treatment for the patient, starting a treatment, etc. Such methods can also be used to monitor treatment of a patient, especially to detect or measure new bone growth. In such embodiments, a first scan may be performed around the time of starting a treatment. Subsequent scans may be used to monitor treatment(s), altering one or more treatments, and other applications mentioned elsewhere in this specification.

FIG. 2J-2L show the steps of a method for comparing bone images from two time points. FIG. 2J shows a first (e.g., baseline) scan of a region of bone. In this particular example, a phalangeal bone is used to demonstrate the method steps. FIG. 2K shows a subsequent scan of the same region of bone wherein the same view of the same bone has been obtained. One or more artifacts were identified in the scan shown in FIG. 2K. Examples of artifacts include, but are not limited to: radiopaque objects implanted in the patient (e.g., medical implants), radiopaque objects external to the patient (e.g., jewelry, piercings), clothing, artifacts from debris in the system or on tissue, soft tissue changes, bone spurs, anatomical abnormalities, noise generated during imaging or processing, fractures, etc. Artifacts may be detected and optionally digitally subtracted from one or more images used for any of the analyses disclosed herein. Area(s) containing artifacts may be excluded from one or more scans used for comparison or other analysis. FIG. 2K shows two artifacts that have been masked (region A, region B). FIG. 2L shows the image of FIG. 2J wherein the same masks of FIG. 2K have been applied to the same regions in FIG. 2J. One or more method steps may be performed using the processed image shown in FIG. 2L. In the particular embodiment shown in FIGS. 2J-2L, the same bone shown in FIG. 2J was subjected to a loss of bone minerals as seen in FIG. 2K. This example is being used to demonstrate the progression of osteoporosis or other diseases causing progressive bone loss. Such regular monitoring of bone loss may be used to determine clinical decisions including, but not limited to: determining an individualized treatment for the patient, starting a treatment, etc. Such methods can also be used to monitor treatment of a patient, especially to detect or measure new bone growth. In such embodiments, a first scan is performed around the time of starting a treatment. Subsequent scans may be used to monitor treatment(s), alter one or more treatments, and other applications mentioned elsewhere in this specification.

One or more methods and devices disclosed herein may be used to monitor patients with osteopenia. Sequential scanning (e.g., every 2 years) may be used to track bone loss and determine progression of bone loss. In some embodiments, such methods may be used to determine progression of osteopenia to osteoporosis.

Any of the changes shown in the bones in FIGS. 2A-2L may be determined using one or more methods and devices disclosed herein. Any of the changes shown in the bones in FIGS. 2A-2L may be used while building one or more ML models.

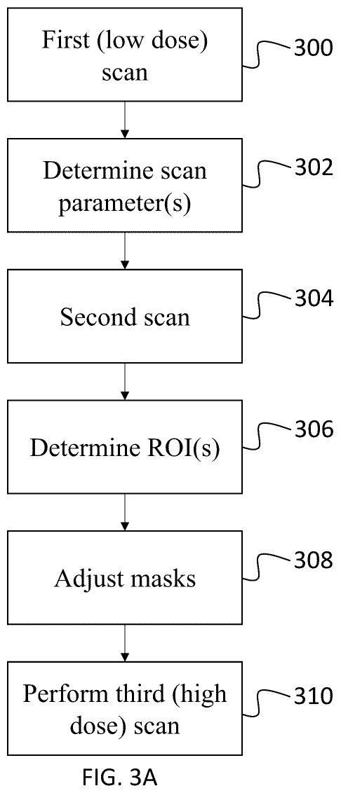

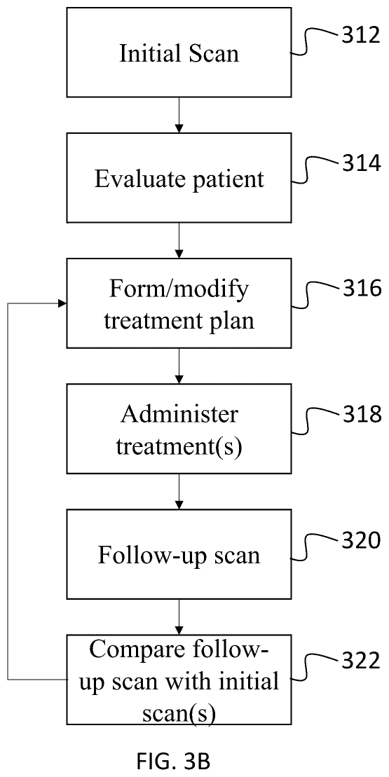

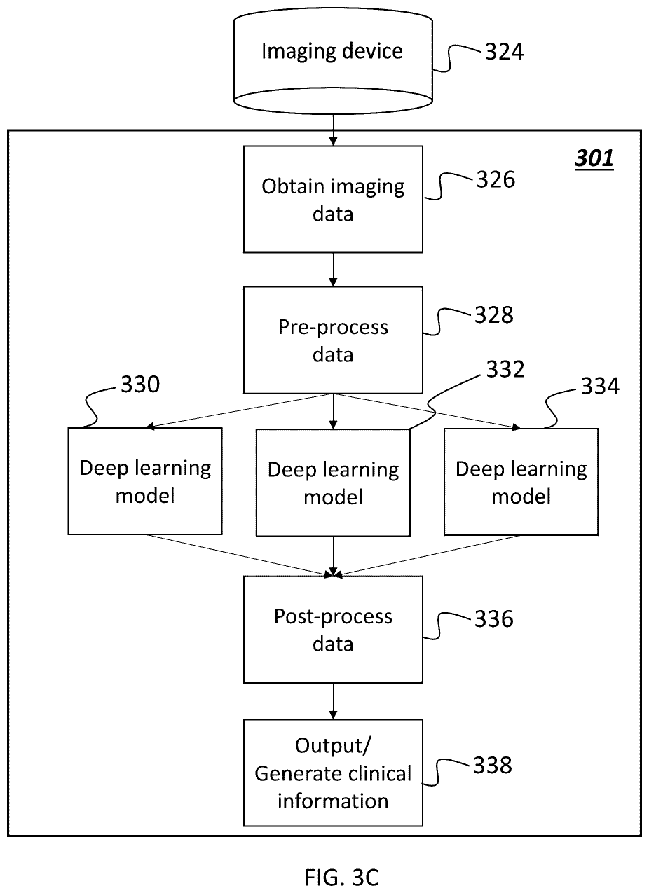

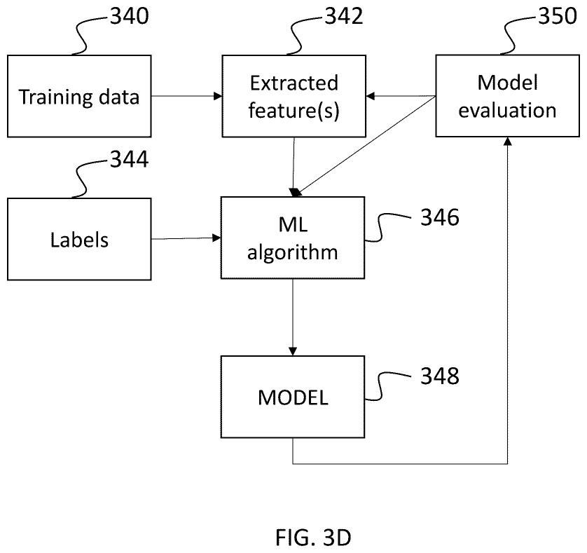

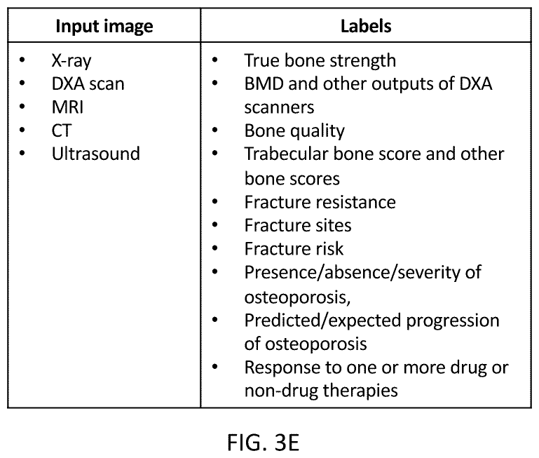

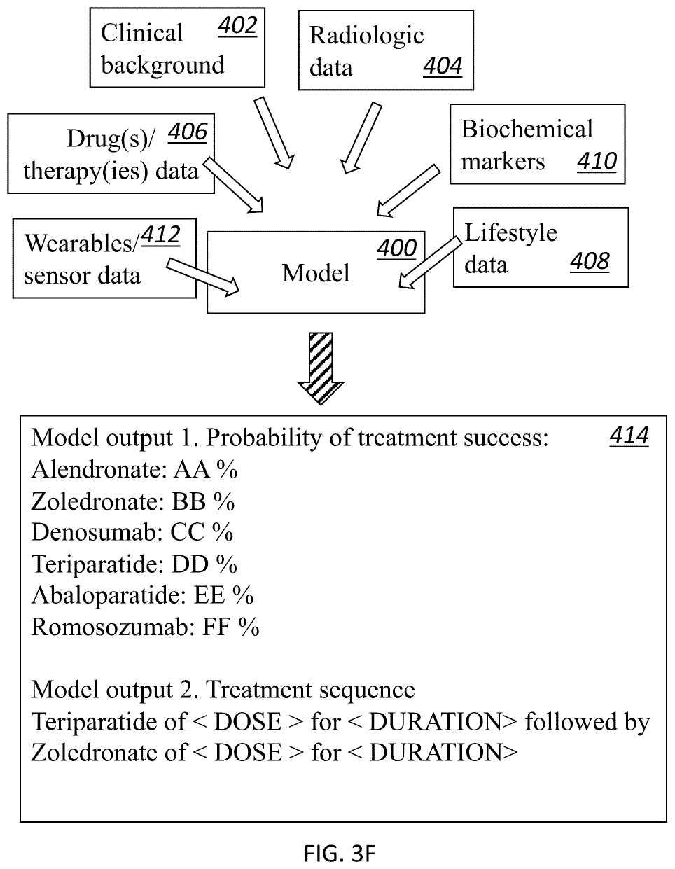

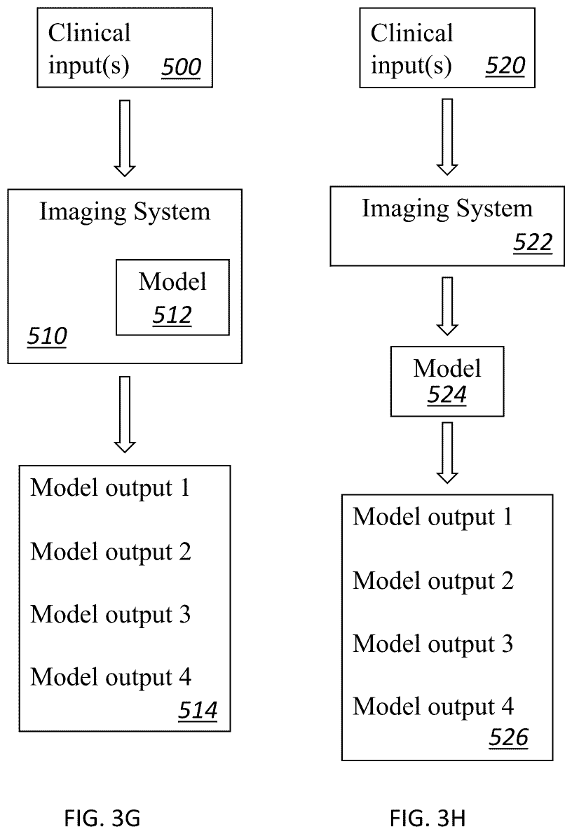

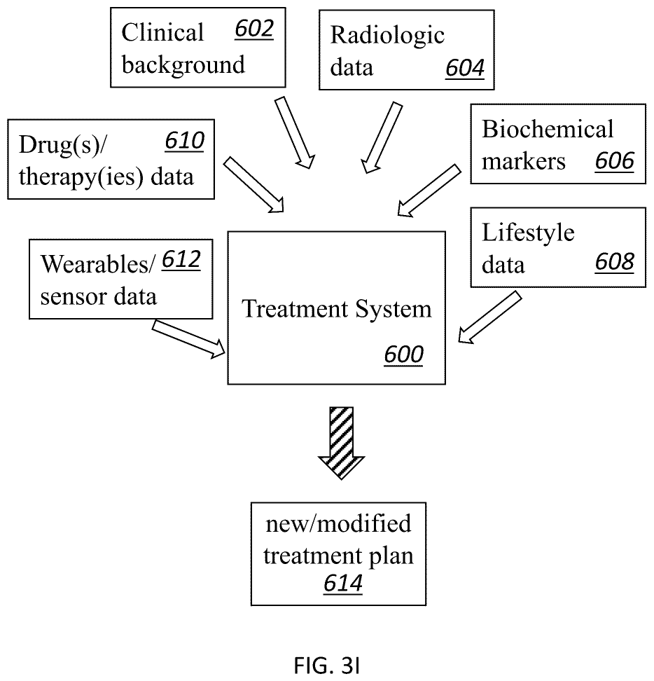

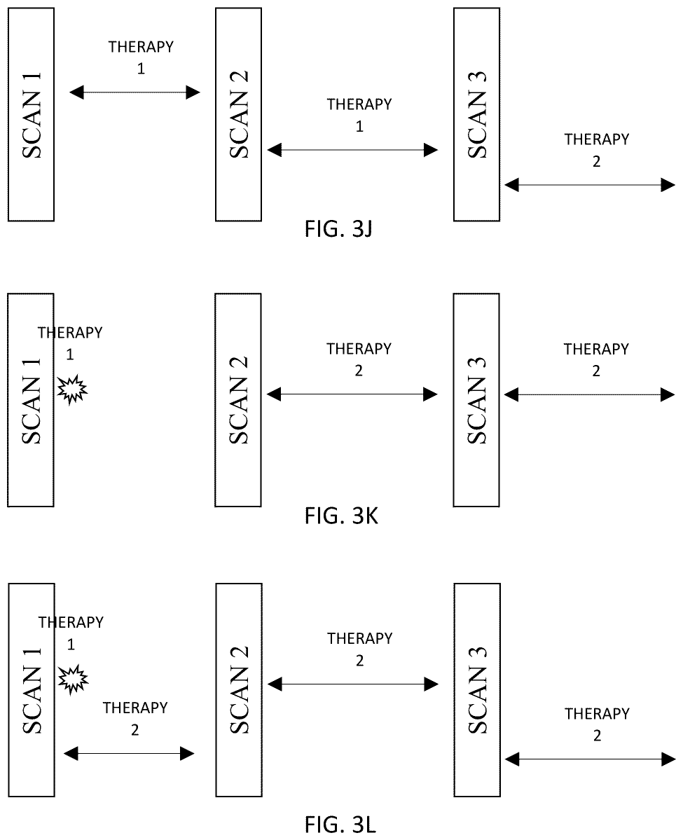

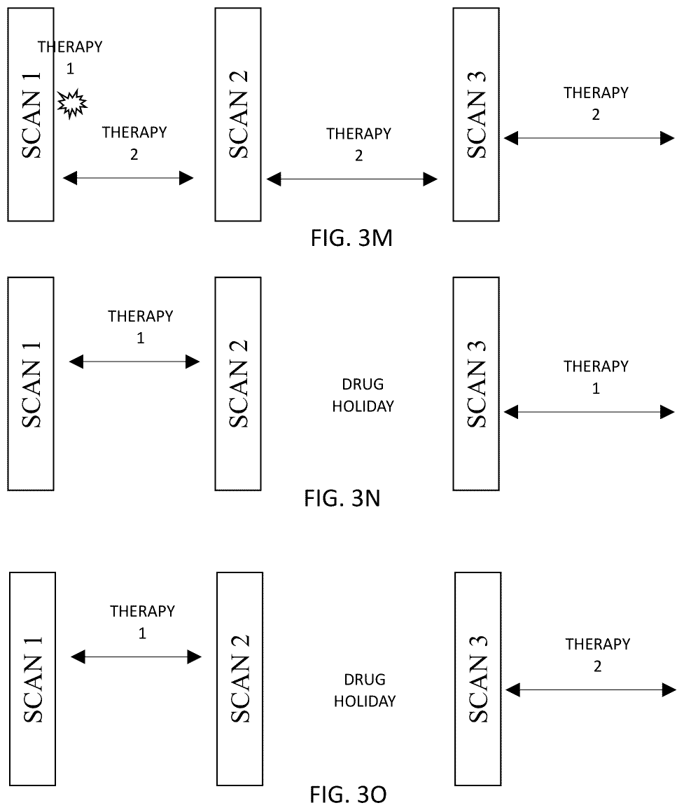

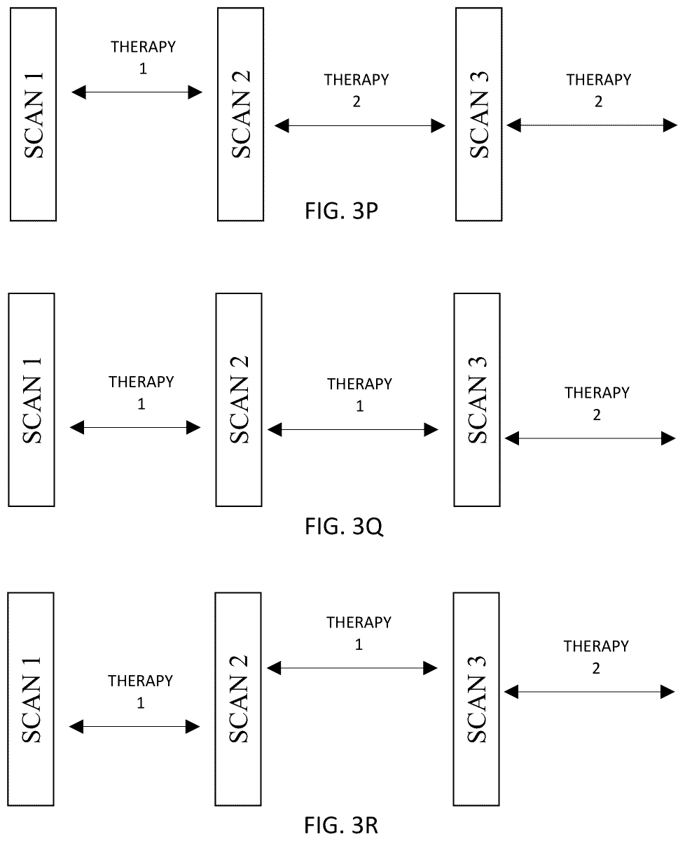

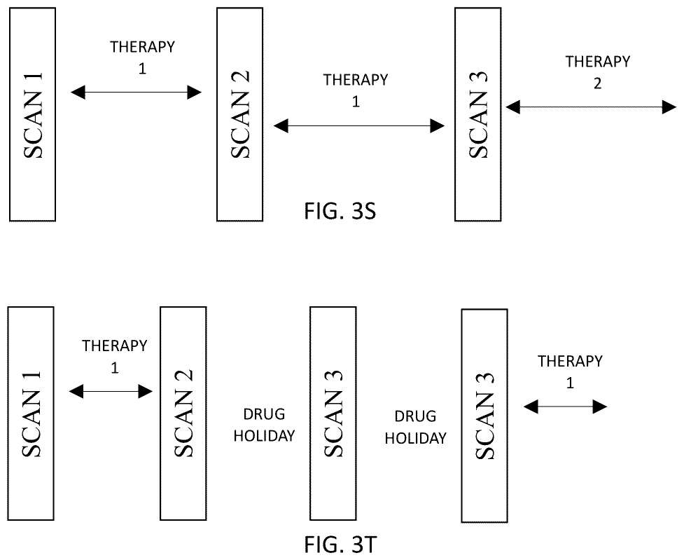

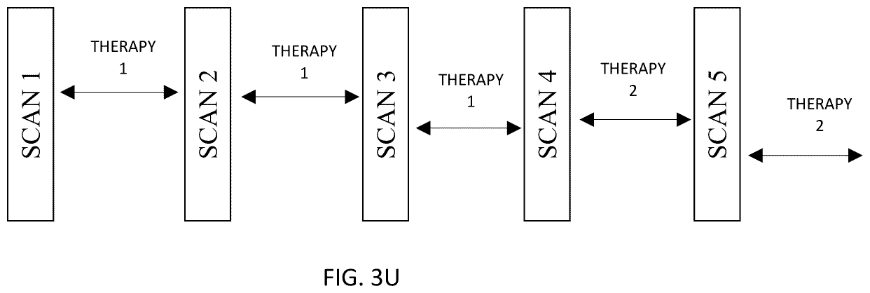

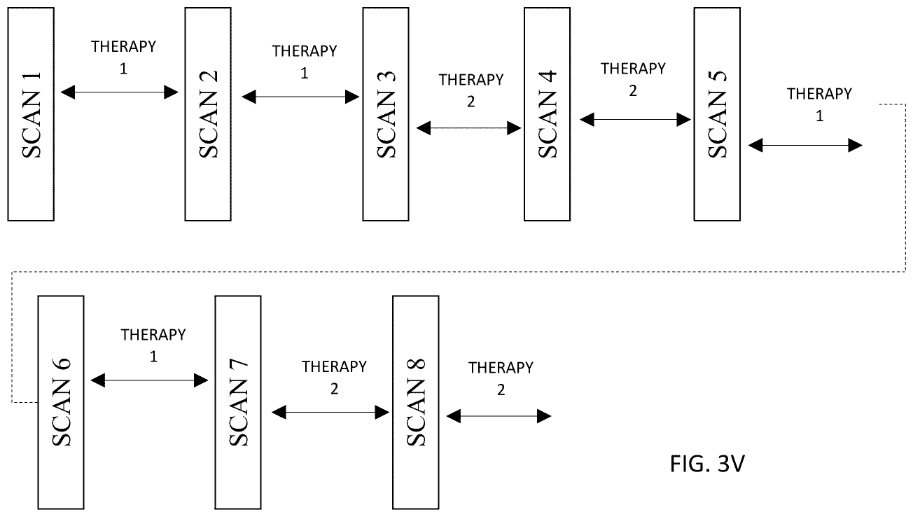

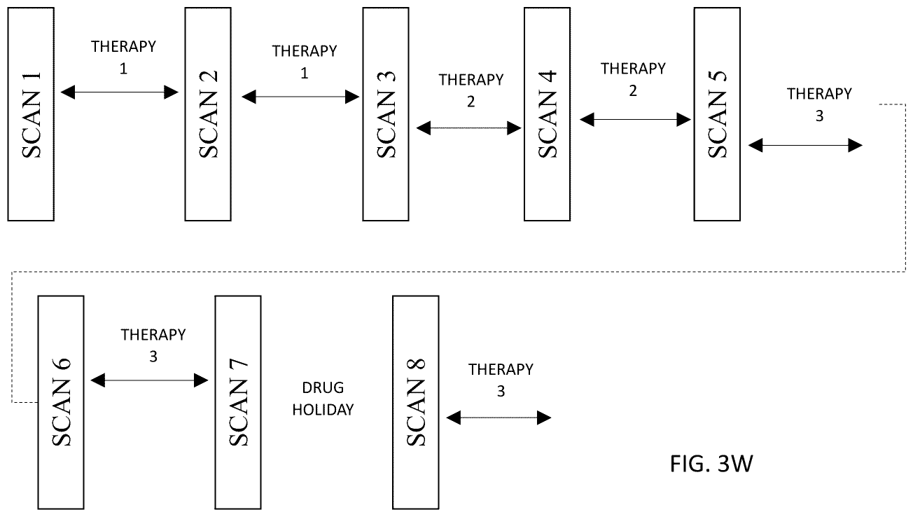

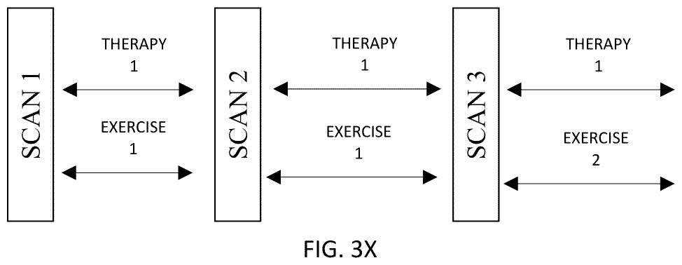

FIGS. 3A-3C shows embodiments of methods of the present disclosure. One or more method steps or sequence of methods steps of some embodiments may be combined with one or more method steps or sequence of methods steps of another embodiment.

FIG. 3A shows an embodiment of a method of the present disclosure wherein the target tissue or organ is scanned at least twice. At step 300, a first scan may be performed. The total radiation dose of the scan may be lower than a subsequent scan. The purpose of this scan is to get a general idea of the bone anatomy in the target tissue or organ. At step 302, one or more scan parameters may be determined. Examples of scan parameters (e.g., resolution, accuracy, orientation, etc.) are disclosed elsewhere in this specification. At step 304, a second scan may be performed. The purpose of this scan is to determine the region(s) of interest in the target tissue or organ. The region(s) of interest include at least one region of a bone. At step 306, one or more masks may be adjusted. In some embodiments, the mask(s) may be adjusted to limit the radiation on tissue that is not of interest. At step 310, a third scan may be performed at a higher radiation dose than the first scan. The data from this scan may be used for one or more applications (e.g., calculating bone strength, bone mineral density and other outputs generated by DXA scanners, calculating one or more bone scores, detecting presence or absence of osteoporosis, predicting response to one or more drug or non-drug therapies, recommending initiating one or more therapies, recommending to not initiate one or more therapies, recommending one or more parameters (e.g. type, dose, route of administration, duration, etc.) of one or more therapies, deciding or recommending timepoints of one or more subsequent scans, deciding or recommending timepoints of one or more subsequent follow-ups, etc.) disclosed herein. Other methods and devices disclosed herein may also be used for the above. If the analysis from this invention predicts a lack of efficacy of a therapy (e.g. a drug therapy such as a bisphosphonate therapy), a user may decide to not initiate that therapy.

FIG. 3B shows an embodiment of a method for regularly monitoring the treatment of a patient and optionally adjusting the treatment plan. At step 312, an initial scan may be performed. This scan may be performed while starting an initial treatment, while screening the patient for treatment suitability, etc. At step 314, the patient may be evaluated. This evaluation may include evaluating one or more inputs including, but not limited to: data obtained from the initial scan, patient's medical history, patient's biochemical markers, etc. Examples of such inputs are disclosed elsewhere in this specification. At step 316, an initial treatment plan may be formed or an existing treatment plan may be modified. Examples of modifying an existing treatment plan are disclosed elsewhere in this specification, including in FIGS. 3J-3X. At step 318, the treatment may be administered to the patient. Thereafter, at step 320, a follow-up scan may be performed. The data from the follow-up scan at step 320 may be compared with one or more previous scan(s). The method can return to step 316.

FIG. 3C shows an embodiment of a method of generating clinical information using one or more deep learning models. At step 324, an imaging device may be used to scan a patient. Examples of imaging devices include, but are not limited to: DXA scanners, X-ray imaging devices, MRI scanners, CT (including micro-CT) scanners, ultrasound scanners, and any of the devices disclosed herein. At step 326, imaging data may be obtained from the imaging device. At step 328, the imaging data may be pre-processed. Examples of pre-processing steps and techniques are discussed elsewhere in this specification. They include, but are not limited to: cropping one or more images, segmenting the one or more images based on one or more parameters, feature extraction, etc.

Segmentation or block definition may be performed in any of the images disclosed herein. Segmentation may be performed manually or using computer-based methods (e.g., using convolutional neural networks like U-Net). Examples of segments include, but are not limited to: bone of interest, trabecular bone, cortical bone, bone margins, soft tissue, regions with similar bone mineral density levels, regions with similar radio-opacity levels, etc. Any of the analyses disclosed herein may be performed on individual segments, or combination of segments. In some embodiments, one or more analyses disclosed herein may be performed solely on trabecular bone. In some embodiments, one or more analyses disclosed herein may be performed solely on cortical bone. In some embodiments, one or more analyses disclosed herein may be performed solely on soft tissue. Examples of segmentation methods include, but are not limited to:
1. Object-oriented approaches comprising several stages starting with the most general objects to be segmented, such as bone outlines from a background, and proceeding in a succession of stages to the most specific object(s), such as trabecular bone. Each stage may use custom operators unique to the needs of that specific stage for increased accuracy.
2. Approaches using deep leaning. For example, using convolutional neural networks (CNNs) such as SDResU-Net, MD-LSTM, U-Net, RFCN, 2D CNN, DeepLab, FCN, etc.
3. Entropy-based segmentation techniques
4. Region-based algorithms in which the regions are constructed using a growing procedure with two or more statistical tests. Tissue classification procedures may be employed following the growing process.
5. Phase based atlas registration
6. Thresholding. This method separates regions in an image based on intensity values above or below a specified threshold.
7. Active contours techniques. In these techniques, deformable models may be used that evolve to fit object boundaries by minimizing an energy functional.
8. Watershed Segmentation wherein an image is treated as a topographic map and segmented based on watershed lines.
9. Methods that track evolving interfaces over time
10. Clustering methods
11. Graph-Cut Segmentation wherein an image is modeled as a graph and segmented by locating the optimal cut based on pixel affinities.
12. Markov Random Fields
13. Atlas-Based Segmentation which involve registering a template or atlas to the target image and then transferring the segmented regions from the template to the target.

The pre-processed data may be fed to one or more artificial intelligence-based methods such as deep learning or other machine learning (ML) models. In several embodiments of this invention, machine learning techniques are used examples of artificial intelligence-based methods.

However other artificial intelligence-based methods such as computer vision-based algorithms, image segmentation algorithms, Fourier transforms and other transforms, fuzzy logic-based methods may also be used. Such methods may be used in conjunction with machine learning. In this embodiment, the data may be sent to three ML models. The output from one or more ML Models may then be post-processed as shown in step 336. Examples of post-processing steps include, but are not limited to: displaying one or more output data superimposed on one or more input data, highlighting the areas of new bone growth, highlighting the areas of new bone loss, etc. At step 338, clinical information may be generated. This may be done by combining the output of any of the methods and devices disclosed herein with one or more additional clinical data. Examples of clinical data are disclosed elsewhere in this specification, but may include, but are not limited to: patient preference, clinical background data, radiologic data (which may be obtained from one or more device and/or methods disclosed herein), biochemical markers, lifestyle data, wearables data, sensor data, drug(s) data, therapy(ies) data, etc.

In any of the embodiments disclosed herein, a machine learning model may be used to perform one or more tasks, examples of which include, but are not limited to: predicting the treatment outcomes of one or more treatments and/or natural clinical course of the patient without interventions. Such models may be used in any suitable system or method embodiments disclosed herein. Such models may be built or trained using one or more images and one or more clinical data. Several examples of imaging modalities used to generate the images are disclosed elsewhere in this specification. They include radiological modalities such as DXA, X-rays, MRI, CT, ultrasound, etc. Examples of clinical data are also disclosed elsewhere in this specification. Clinical data e.g. clinical background data, biochemical marker(s) data, lifestyle data, wearables data, sensor data, drug(s) data, therapy(ies) data, etc. may be used as metadata for the images used to build or train the ML models. Examples of methods used to build such ML models include, but are not limited to:
1. Convolutional Neural Networks (CNNs): CNNs can be used to automatically extracting features relevant to the prediction task. Examples of CNNs include, but are not limited to: AlexNet, VGGNet, ResNet, and Inception. Metadata may be incorporated into CNN models through one or more methods such as fusion with image features, parallel processing pipelines, etc.
2. Vision Transformer (ViT) model architectures that leverage the transformer model.
3. Deep neural networks, wherein regions of interest (ROIs) are extracted from an image by a rotated object detection technique used in region proposal networks.
4. Deep neural networks, wherein anatomical landmarks are detected and used for extracting ROIs. One or more calculations and functions disclosed herein may be performed on the extracted ROIs.
5. Prediction models for predicting one or more bone parameters such as bone strength, bone density, bone growth, etc. based on deep convolutional neural networks.
6. Graph Neural Networks (GNNs): GNNs may be used to model relationships between patients based on metadata (e.g., patient similarity graphs). Image data may be incorporated as node features.
7. Deep Learning Architectures using Multi-Modal Fusion: Multi-Modal Neural Networks (MMNNs) or multi-input and/or multi-output networks may be used to learn joint representations of image and metadata data, thereby allowing for multiple interactions between such data.
8. Transfer Learning: In such methods, pre-trained deep learning models (e.g., CNNs) may be fine-tuned on medical image datasets along with patient metadata. In transfer learning methods, knowledge from large datasets may be used to improve performance on smaller, domain-specific datasets.
9. Support Vector Machines (SVMs): Extracted features from both images and metadata may be used as inputs to SVM models. SVMs may be used for classification tasks.
10. Random Forests and Gradient Boosting Machines: Ensemble learning methods like Random Forests and Gradient Boosting Machines may be used to process multiple data types and capture complex interactions between features. Such methods may incorporate image features extracted using techniques such as histogram of gradients as well as features of metadata.
11. Recurrent Neural Networks (RNNs): RNNs can be used for sequential data modeling, such as time-series data or sequential patient records. Metadata can be treated as sequential data (e.g., temporal changes in patient vitals), while images may be processed using techniques such as CNNs.
12. Ensemble models may be used to perform one or more classifications and or calculations disclosed herein. In such ensemble models one or more additional inputs may be provided. Examples of such additional inputs include, but are not limited to: details of the drug therapy being administered to the patient, bone mineral density data, other imaging data, the patient demographic data, data on the size and other parameters of the organ or tissue being imaged, data obtained through a digital health or consumer device, etc. Details of the drug therapy include but are not limited to: type of drug, route of administration, dosage, dosage history, duration of treatment, date(s) of Initiation of treatment, date(s) of completion of treatment, etc.
13. Hybrid Approaches: One or more of the above methods may be combined. E.g. CNNs may be combined with RNNs for processing sequential data along with images.

Any of the ML models disclosed herein may be trained using one or more clinical endpoints or labels including, but not limited to: presence or absence of osteoporosis, fracture risk, presence or absence of crack propagation sites, BMD, response to therapy, true bone strength (which may be calculated e.g., through Finite Element Analysis-FEA), bone quality, trabecular score, bone strength parameter(s) determined using human cadaver bones, etc. Bone strength testing (e.g., resistance to impact, resistance to crack propagation) or other tests may be performed on cadaver bones and the data may be fed to or otherwise used by one or more device or method embodiments herein. The training may be performed using supervised machine learning, wherein labeled datasets are used to train algorithms that classify data or predict outcomes. After input data is fed into a model, the model may adjust its weights until the model has been fitted appropriately as determined through a cross-validation process. These clinical endpoints may be determined by a computer-based or human analysis and used to label one or more training datasets. In one such example, one or more processed images (examples include, but are not limited to: images taken at baseline, images taken after administering a therapy, images showing a change relative to time, etc.) are fed to a ML model along with one or more clinical endpoints (labels) such as bone strength. The bone strength may be determined through methods including, but not limited to: CT scanning, MRI scanning, bone biopsy analysis, FEA, etc. The ML model is then trained to classify and/or score one or more input images to determine actual or predicted changes in bone strength.

In some embodiments, the system identifies two or more regions of interest of the imaged tissue (e.g., one or more regions of one or more bones). The system performs data processing on two or more regions of interest using two or more methods or models. In such embodiments, a first machine learning model (e.g., ResNet) may be used to analyze a cortical region of interest and a second machine learning model (e.g., EfficientNet) may be used to analyze a trabecular region of interest.

For any of the machine learning models disclosed herein, a Multiple-blocks regression strategy may be used. In this strategy, the image is divided into multiple blocks. This may be done using anatomical markers and/or measurements and/or segments. The one or more blocks may be one-hot-encoded and further processing may be performed.

FIG. 3D shows an example of a flow chart of a supervised ML model that uses labeled data. Training data 340 is cleaned or otherwise pro-processed and fed to a feature extraction module to extract one or more useful extracted feature(s) 342 from the data. The one or more labels 344 or known class along with extracted feature(s) 342 may be passed to the training phase where machine learning algorithms 346 may be used to identify a particular model which map inputs to desired outputs. Model evaluation 350 may be used to provide feedback to feature extraction and learning phases. The extracted feature(s) 342 and/or ML algorithm(s) may be adjusted to improve accuracy of ML model 348. The training process may be repeated one or more times until a desired accuracy level is achieved. The ML model 348 may then be used to perform one or more methods disclosed herein, e.g., to predict the labels of data obtained from an imaging device.

In some embodiments, ML model 348 may be trained using an image of a bone region obtained through an imaging modality, examples of which are disclosed elsewhere in this specification. Labels 344 include one or more bone strength parameters, examples of which include, but are not limited to BMD, true bone strength, fracture resistance, fracture risk, outputs of DXA scanners, etc. Trained ML model may be used for predicting the bone strength parameters based on an input image. One advantage of this method may be that bone strength parameters such as BMD or any of the parameters that can only be produced currently using DXA scanners can be obtained using simple imaging modalities such as plain X-ray of bones such as spine, hip bones, peripheral long bones, etc.

In some embodiments, a trained ML model may be used for predictive analytics to predict the response of a patient to one or more drug or non-drug therapies. In some embodiments, a trained ML model may be used for predictive analytics to predict the response of a treatment-naïve patient to bisphosphonate therapy. A predicted lack of sufficient therapeutic response to bisphosphonate therapy may be used to treat the patient using non-bisphosphonate therapies; examples of which are disclosed herein. In another embodiment, a trained ML model may be used for predictive analytics to predict the further response of a patient to an ongoing therapy. A predicted lack of sufficient continuing therapeutic response may be used to switch the patient to another therapy; several examples of which are disclosed herein.

FIG. 3E shows a list of selected imaging methods and labels of the present disclosure. Multiple embodiments of the present disclosure may be generated or trained by using one or more suitable labels with a suitable imaging method. In one embodiment, one or more devices and methods disclosed herein are trained using one or more labels that include data on patients' responses to one or more drug or non-drug therapies. Such responses could include data such as efficacy, safety, speed of action, side effects, etc. of the one or more drug or non-drug therapies. Thereafter, a trained model can be used to predict a patient's response to one or more drug or non-drug therapies.

FIG. 3F shows one such machine learning model being used to predict the treatment outcomes of one or more treatments. As shown in FIG. 3F, the model, at block 400, is built or trained using multiple inputs which may include clinical data or background at block 402, radiological data (e.g., one or more images) at block 404, biochemical markers at block 410, lifestyle data at block 408, drug(s) and/or therapy(ies) data at block 406, and/or wearable and/or sensor data at block 412. A first output of the model may be a prediction of the probability of treatment success of a variety of therapies, as shown at block 414 in FIG. 3F. The treatment success in any of the embodiments herein may be defined in a variety of ways, examples of which include, but are not limited to: increase in BMD, increase in fracture resistance, strength parameter(s) determined through computational methods, strength parameter(s) determined through imaging, reduction in fracture risk, etc. A second output of the model may be a sequence of treatments, one example of which is shown in FIG. 3F.

FIGS. 3G and 3H show two method embodiments showing how an AI-based model of the present disclosure may be incorporated in a treatment method using an imaging system. In the method shown in FIG. 3G, clinical inputs at block 500 are fed to an imaging system at block 510 incorporating an AI-based model at block 512. Examples of imaging systems and AI-based models are disclosed elsewhere herein. The imaging system then produces one or more outputs at block 514; examples of which are given elsewhere in this specification. In the method shown in FIG. 3H, clinical inputs at block 520 are fed to an imaging system at block 522. Thereafter, the output of the imaging system is fed into one or more AI-based models at block 524. Examples of imaging systems and AI-based models are disclosed elsewhere in this specification. The AI-based model then produces one or more outputs at block 526, examples of which are given elsewhere herein.

FIG. 3I illustrates how multiple input parameters can be considered along with embodiments of the present disclosure (e.g. trained ML models, imaging systems, etc.) to determine the treatment plan of a patient at block 600. As shown, examples of the input parameters include, but are not limited to: clinical background at block 602, radiologic data at block 604 (which may be obtained from one or more devices and methods disclosed herein), biochemical markers at block 606, lifestyle data at block 608, wearable data and/or sensor data at block 612, drug(s) data and/or therapy(ies) data at block 610, etc. The treatment plan or system at block 600 may be updated based on the various inputs so that a new or modified treatment plan is output from the system at block 614.

Clinical background data includes, but is not limited to, risk factors such as: low body mass index (BMI), prior fracture history especially at regions that are at high risk for osteoporotic fractures, parental hip fracture history, drug use (e.g., glucocorticoid use) history, smoking history, and alcohol consumption history. Wearable data and/or sensor data includes, but is not limited to:
1. Application of weight and/or impact to bones e.g., through weight bearing exercises (these may be used to stimulate osteoblast activity)
2. Measurement(s) of balance
3. Detection and/or measurement(s) of balance issues
4. Measurement(s) of gait and/or posture when walking
5. Measurement(s) of activity level
6. Measurement(s) of mechanical impacts
7. Measurement(s) of slip and/or off-balance events

Measurement(s) of gait and/or posture include, but are not limited to: measure(s) of unstable gait, data from apps such as WalkAI® developed by Zimmer Biomet, AI based gait analyses, data from VR based technologies like GaitBetter, etc. Any data generated by one or more embodiments may be used as inputs for therapies to train based on gait, teach based on gait, correct gait, detect gait abnormality, etc.

In some embodiments, the wearable is the sleep tracking smart ring “Oura Ring” made by Ōura Health Oy. Wearables may measure parameters such as sleep, temperature, movement, exercise, heart rate, blood oxygen, falls, etc.

Any of the inputs used in any version of Fracture Risk Assessment Tool (FRAX®) developed by University of Sheffield or other fracture risk assessment tools may be used as input parameters for any of the methods and systems disclosed herein. Further, any of the data generated by the present disclosure may be used as input(s) for one or more fracture risk assessment tools including, but not limited to: modifications of Fracture Risk Assessment Tool (FRAX®). In some embodiments, a score output of a fracture risk assessment tool is modified based on any of the data (e.g., bone changes) generated from one or more device and/or methods disclosed herein. This is especially useful for assessing fracture risk in patients who are on treatment. In some embodiments, a first baseline scan may be performed at the time of starting a therapy and a first baseline fracture risk may be assessed. A second scan may be performed after completing at least a portion of the therapy. One or more inputs may be provided to a fracture risk assessment tool and a second post-treatment fracture risk may be assessed. Examples of such inputs include, but are not limited to: any of the data generated from one or more devices and methods disclosed herein, change(s) in biochemical parameter(s), inputs from wearables or other monitoring devices (e.g. balance, activity, falls, gait, etc.), etc. In some embodiments, any of the data generated by the present disclosure may be used as input(s) for a fracture risk assessment tool after starting a bone modifying therapy (e.g., osteoporosis therapy).

Data regarding one or more biochemical markers may be used in any of the methods disclosed herein to determine one or more parameters (e.g., type of drug, dose of drug, start or stop drug therapy, etc.) disclosed herein. Biochemical markers include, but are not limited to: bone turnover markers (BTMs). Examples of BTMs include, but are not limited to:
1. Bone formation markers. Examples include, but are not limited to: serum procollagen type I N-terminal propeptide (PINP) —an osteoblast marker, C-terminal propeptide of type I procollagen (PICP), Osteocalcin, Bone-specific alkaline phosphatase (BAP, bone ALP), etc.
2. Bone resorption markers. Examples include, but are not limited to:
2A. Collagen-related markers such as serum carboxy terminal telopeptide of collagen type I (s-CTX), serum carboxy-terminal collagen crosslinks (CTX), Hydroxyproline-total and dialysable (Hyp), Hydroxylysine-glycosides, Pyridinoline (PYD), Deoxypyridinoline (DPD), Aminoterminal cross-linked telopeptide of type I collagen (NTX-I), Collagen I alpha 1 helicoidal peptide (HELP), etc. CTX is released directly from bone from osteoclastic resorption.
2B. Non-Collagenous Proteins such as Bone Sialoprotein (BSP), Osteocalcin fragments (ufOC, U-Mid-OC, U-LongOC), Osteoclast Enzymes etc. Osteoclast Enzymes include Tartrate-resistant acid phosphatase (TRAcP), Cathepsins (e.g. K, L), etc.

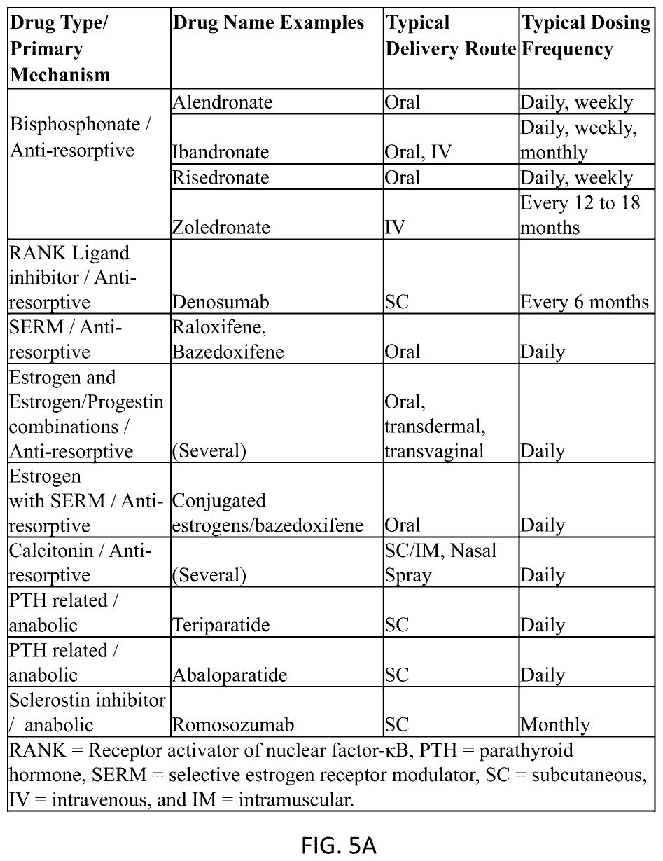

One or more methods and/or one of more devices (e.g., analysis system) described herein may be used to adjust one or more osteoporosis therapies. Such therapies include, but are not limited to: drugs, exercises, lifestyle modifications, vibration-based therapies, gait-based therapies, activity-based therapies, and combinations thereof. For example, osteoporosis therapies may be designed using exercise(s) plus osteoanabolic drugs. Adjustment of a drug may include one or more of: introduction of a drug treatment, discontinuation of a drug treatment, changing the dose of a drug, changing the route of administration of a drug, changing the setting of a drug delivery system, changing the typical administration parameters (e.g., dose per administration, dosing frequency, duration, delivery route, etc.), transitioning to a different therapy, etc. One or more parameters related to such adjustment e.g., transition time-point between therapies, may be determined by the devices and methods described herein. Examples of drugs include, but are not limited to: oral drugs such as bisphosphonates (e.g., alendronate, risedronate, and ibandronate), estrogen, and Raloxifene; intravenous drugs such as bisphosphonates and Zoledronic acid; drugs administered as injections such as Denosumab, estrogen, Teriparatide, and Calcitonin; drugs administered as nasal sprays such as calcitonin; drugs administered as skin patches e.g. estrogen; drugs injected into the intestinal wall e.g., RaniPill™ injecting PTH; and drugs administered as gels e.g. estrogen. FIG. 5A lists examples of drugs classes, drugs, and their typical delivery routes, and typical dosing frequency. Any of the parameters (e.g., drug type or class, dosing frequency, delivery route, dose, start or end of a drug holiday, etc.) may be modified based on one or more method or device embodiments disclosed herein. The duration of administration of a drug or other therapy may also be modified based on one or more method or device embodiments disclosed herein. For example, romosozumab is typically administered for 12 months. That typical duration may be modified such that romosozumab is administered for less than about 11 months or for less than about 13 months. In another, teriparatide and abaloparatide are typically administered for about 24 months. That typical duration may be modified such that they are administered for less than about 22 months or for less than about 26 months. Thus, the risk or benefit profile of a therapy can be optimized for an individual patient using the present disclosure.

In FIGS. 3J-3X, the vertical level of the therapy shows the amount (e.g., total dose per administration, total dose per unit time, etc.) of the therapy and the horizontal extent shows the time. One or more steps or sequences shown in FIGS. 3J-3X may be combined with other steps or sequences shown in FIGS. 3J-3X.

FIG. 3J shows an embodiment of a method of the present disclosure that includes two therapies wherein a therapy parameter of a first therapy is adjusted. In FIG. 3J, a first scan (Scan 1) may be performed and first therapy (Therapy 1) may be started. In any of the embodiments herein, one or more parameters of the first therapy may be determined using the data and information generated from the first scan. Examples of such parameters include, but are not limited to: initiation or non-initiation of therapy, type of therapy, therapy dose, route of administration, therapy duration, timepoint of subsequent scan(s), timepoint of subsequent follow-up(s), etc.

Examples of scans and therapies are disclosed herein. Scan 2 may be performed and one or more parameters disclosed herein are determined. A dose parameter (examples disclosed herein) may be lowered. Scan 3 may be performed. After Scan 3, Therapy 1 may be discontinued and Therapy 2 may be started. Therapy 1 may be discontinued for reasons including, but not limited to: loss or tapering of effect, one or more side-effect(s), achieve of peak therapeutic effect, lowering of rate of bone gain, etc.

FIG. 3K shows an embodiment of a method of the present disclosure that includes two therapies wherein the first therapy may be administered as a bolus. Examples of Therapy 1 include, but are not limited to, therapies like zoledronate, anti-RANKL antibodies such as denosumab, anti-sclerostin antibodies such as romosozumab, etc. After Scan 2, Therapy 1 may be discontinued for any of the reasons disclosed herein and Therapy 2 may be started. At Scan 3, it may be determined that Therapy 2 may be continued as is and Therapy 2 may be continued for another time period.

FIGS. 3L and 3M show more embodiments of a method of the present disclosure that includes two therapies wherein the first therapy may be administered as a bolus. Examples of Therapy 1 include, but are not limited to, therapies like zoledronate, anti-RANKL antibodies such as denosumab, anti-sclerostin antibodies such as romosozumab, etc. After Scan 2, Therapy 1 may be discontinued for any of the reasons disclosed herein and Therapy 2 may be continued at the same or higher or lower dose parameters. At Scan 3, it may be determined that a dose parameter of Therapy 2 may be changed, and Therapy 2 may be continued thereafter for another time period with the changed dose parameter.

FIGS. 3N and 3O shows method embodiments of the use of the present disclosure for determining and/or managing a drug holiday. Therapy 1 may include a bisphosphonate drug. After Scan 2, a drug holiday (a period when the drug is not administered) may be started. A drug holiday may be started based on the results of Scan 2. A drug holiday may be started for one or more reasons including, but not limited to: adverse event(s), finding(s) from imaging data, detecting early changes indicative of adverse effects, lowering the risk of AFF, lowering the risk of ONJ, lowering fracture risk from treatment with Therapy 1, determining that adequate response to Therapy 1 has been achieved, etc. After Scan 3, Therapy 1 may be restarted at the same or different dose than before (as shown in FIG. 3N). The post-holiday therapy could be a different bisphosphonate, or a non-bisphosphonate as shown in FIG. 3O. The treatment may be restarted based on the results of Scan 3. The treatment may be restarted for one or more reasons including, but not limited to: increased fracture risk, significant loss of bone mineral or bone strength, etc.

FIG. 3P show another embodiment of a method of the present disclosure comprising two therapies. In one such embodiment, Therapy 1 may be an osteoanabolic therapy and Therapy 2 may be an anti-resorptive therapy; examples of both are disclosed herein. In another embodiment, Therapy 1 may be an anti-RANKL antibody such as denosumab. Patients who are stopping denosumab can be transitioned to another therapeutic agent. This may occur when long-term denosumab therapy is stopped, since it will likely be followed by loss of BMD, increase and overshoot of bone turnover markers, and increase of fracture risk. Therapy 2 may be an anti-resorptive therapy.

FIG. 3Q shows a method embodiment of sequencing two therapies. Scan 1 may be performed at the initiation of Therapy 1. The effect of Therapy 1 may be tracked using Scan 2. Therapy 1 may be continued for another duration and the effect of Therapy 1 may be again tracked using Scan 3. At Scan 3, it may be determined that Therapy 1 may be replaced with Therapy 2. This may be done, for example, to achieve an increased therapeutic effect, to shift from an anabolic therapy to an anti-resorptive therapy, to shift from an anti-resorptive therapy to an anabolic therapy, etc. In one particular embodiment, Therapy 1 may be a bisphosphonate therapy and Therapy 2 may be started when Therapy 1 is not showing a desired effect or is predicted to achieve a low therapeutic effect. Transitioning to Therapy 2 may be used to increase fracture protection in the patient.

FIG. 3R shows another method embodiment of sequencing two therapies. In this embodiment, the dose of Therapy 1 may be increased after Scan 2. However, it is found that even with the increased dose, Therapy 1 is not showing a desired effect. This is determined using Scan 3. Thereafter, Therapy 1 may be stopped and Therapy 2 may be started.

FIG. 3S shows another method embodiment of sequencing two therapies. In this embodiment, the dose of Therapy 1 may be reduced after Scan 2. At Scan 3, it may be determined that the patient may be transitioned to Therapy 2. In some embodiments, Therapy 1 may be an anabolic whose one or more dose parameters are reduced after Scan 2. This may be done for reasons including, but not limited to: reducing short term side effects, reducing potential long term side effects, reducing the effect is an excessive effect is seen, completion of a course of anabolic therapy, etc. Therapy 2 may be an anti-resorptive therapy. In any of the method embodiments disclosed herein, an anabolic therapy may be followed by anti-resorptive therapy (e.g., bisphosphonates, denosumab) to preserve and possibly further increase bone material or bone strength gains.

FIG. 3T shows another method embodiment of the use of the present disclosure for determining and/or managing a drug holiday. Therapy 1 may include a bisphosphonate drug. After Scan 2, a drug holiday (a period when the drug is not administered) may be started. A drug holiday may be started based on the results of Scan 2. A drug holiday may be started for one or more reasons including, but not limited to: imaging data, lowering the risk of AFF, lowering the risk of ONJ, low fracture risk after treatment with Therapy 1, adequate response to Therapy 1, etc. After Scan 3, it may be determined that the period of the drug holiday can be safely extended. This may be done for reasons including, but not limited to: low rate of bone loss, maintenance of increased bone mass, etc. The drug holiday may be monitored using Scan 4. After Scan 4, Therapy 1 may be restarted at the same or different dose than before. The treatment may be restarted based on the results of Scan 4. The treatment may be restarted for one or more reasons including, but not limited to: increased fracture risk, significant loss of bone mineral or bone strength, etc.

FIG. 3U shows another method embodiment of sequencing two therapies. Therapy 1 and Therapy 2 may be any of the therapies disclosed herein. Scans may be selected from any of the imaging techniques disclosed herein. Any of the changes or transitions in one or more therapies may be performed using any endpoint or measurement disclosed herein. In some embodiments, Therapy 1 is a PTH-based drug or other anabolic drug that leads to net anabolic effects on bone and creates newly formed bone. An individualized type, duration, dose etc. of Therapy 1 may be determined using the present disclosure. It may be adjusted at one or more times as shown. As shown after Scan 4, Therapy 1 may be discontinued and Therapy 2 may be administered. In some embodiments, therapy 2 includes an anti-resorptive drug (e.g. a bisphosphonate, a RANKL inhibitor) which decreases the rate of resorption of the newly formed bone. An individualized type, duration, dose, etc. of Therapy 2 may be determined using the present disclosure. In some embodiments, one or more scans performed after administering Therapy 2 may be used to determine if the improvement in therapeutic parameters (examples include, but are not limited to: BMD, bone strength, fracture resistance, etc.) from Therapy 1 may be maintained or even enhanced. In some embodiments, Therapy 2 may be administered for at least 1 year. In some embodiments, Therapy 1 is a PTH-based drug like teriparatide or abaloparatide that is administered for less than about 2 years and Therapy 2 is denosumab. In another exemplary embodiment, Therapy 1 is a PTH based drug like teriparatide or abaloparatide that is administered for less than about 2 years and Therapy 2 is denosumab. In some embodiments, Therapy 1 is an estrogen-based drug like SERM and therapy 2 is an anti-resorptive drug (e.g., a bisphosphonate, a RANKL inhibitor). Therapy 1 may be administered after menopause. In another specific embodiment, Therapy 1 is a combination of a PTH based drug (e.g., teriparatide or abaloparatide) and denosumab and Therapy 2 is denosumab.

FIG. 3V shows another method embodiment of sequencing two therapies. In this embodiment, therapy 1 may include a PTH-based drug or other anabolic drug that leads to net anabolic effects on bone and creates newly formed bone. The dose parameters (e.g., type, duration, dose, etc.) of therapy 1 may be determined using the present disclosure. Thereafter, as shown after Scan 3, Therapy 1 may be discontinued and Therapy 2 may be administered. In some embodiments, Therapy 2 includes an anti-resorptive drug (e.g. a bisphosphonate, a RANKL inhibitor) which decreases the rate of resorption of the newly formed bone. The dose parameters (e.g., type, duration, dose, etc.) of Therapy 2 may be determined using the present disclosure. Thereafter, a Scan 5 may be performed. After Scan 5, Therapy 1 may be restarted and continued even after Scan 6. The reasons for re-starting Therapy 1 include, but are not limited to: loss of bone or bone strength, to increase fracture resistance, etc. Scan 7 may be performed after which Therapy 2 may be restarted. In this way, Therapy 1 (e.g., an anabolic drug) and Therapy 2 (e.g. an anti-resorptive) may be alternated to achieve an individualized treatment of the patient.

One of the advantages of the portable and low-cost imaging techniques disclosed herein is that the patient's bones can be imaged multiple times in the office environment. In some embodiments, one or more scans may be performed at the time of prescribing and/or administering a therapy, for example at the time of prescribing and/or administering drugs like denosumab, romosozumab or zoledronate. In some embodiments, a first therapy is delivered for a first-time duration. During this duration, one or more regions of the patient's anatomy are imaged one or more times. The first therapy may be administered until the effect is tapering or reversing as seen by any of the method or device embodiments disclosed herein. A second therapy may be delivered for a second time duration. During this duration, one or more regions of the patient's anatomy may be imaged one or more times. The second therapy may be administered until the effect is tapering or reversing as seen by any of the method or device embodiments disclosed herein. In this way, the first therapy (e.g., an anabolic drug) and the second therapy (e.g. an anti-resorptive) may be alternated to achieve an individualized treatment of the patient.

FIG. 3W shows a method embodiment of sequencing three therapies. In this embodiment, Therapy 1 may be a bisphosphonate or other first line drug. The system may be used to determine the effects of Therapy 1. At Scan 3, it may be determined that the first line therapy is not going to benefit the patient. Thereafter, the patient may be shifted to Therapy 2. In this embodiment, Therapy 2 may comprise a PTH-based drug or other anabolic drug that leads to net anabolic effects on bone and creates newly formed bone. The dose parameters (e.g., type, duration, dose, etc.) of Therapy 2 may be determined using the present disclosure. As shown after Scan 5, Therapy 2 may be discontinued, and Therapy 3 may be administered. In some embodiments, Therapy 3 includes an anti-resorptive drug (e.g., a bisphosphonate, a RANKL inhibitor, etc.) which decreases the rate of resorption of the newly formed bone. The dose parameters (e.g., type, duration, dose, etc.) of therapy 3 may be determined using the present disclosure. After Scan 7, Therapy 3 may be discontinued for a drug holiday. The drug holiday may be managed as per any of the embodiments disclosed herein. Scan 8 may be performed after which Therapy 3 may be restarted.

FIG. 3X shows a method embodiment of managing multiple, simultaneous therapies. In this case, one of the therapies may be an exercise therapy. Scan 1 may be performed at the initiation of Therapy 1 and/or Exercise 1. Therapy 1 and Exercise 1 may be administered; examples of both are disclosed elsewhere in this specification. The combined effect of Therapy 1 and Exercise 1 may be tracked using Scan 2. One or more parameters of Therapy 1 and/or Exercise 1 may be adjusted or maintained. Both therapies may be continued for another duration and a combined effect of Therapy 1 and Therapy 2 may be again tracked using Scan 3. At Scan 3, it may be determined that Exercise 1 can be replaced with Exercise 2. The combined effect of Therapy 1 and Exercise 2 may then be tracked using any of the methods disclosed herein and one or more adjustments may be made as disclosed herein.

One or more device and/or method embodiments herein may be used to predict the future and/or past changes in one or more bone parameters. In some embodiments, device and/or method embodiments herein may be used for predicting and/or estimating the peak increase in bone strength or other parameters by one or more therapies. In one such embodiment, the therapy may be stopped when the additional risk from the therapy is not worth the small increase in benefits. In some embodiments, changes in parameters (examples of which are disclosed herein) between two time points may be used to determine any of the outcomes listed herein. Examples of such outcomes include, but are not limited to: predicted or expected progression of osteoporosis, therapy response, presence or severity of osteoporosis, initiation of therapy, fracture risk, etc. One or more device and/or method embodiments herein may be used to analyze qualitative features of one or more bone imaging techniques to diagnose osteoporosis or to initiate therapy.

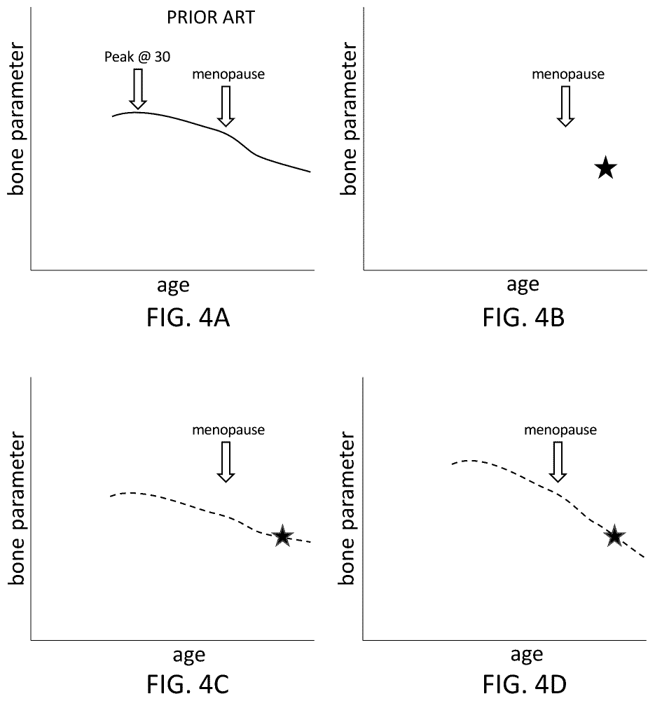

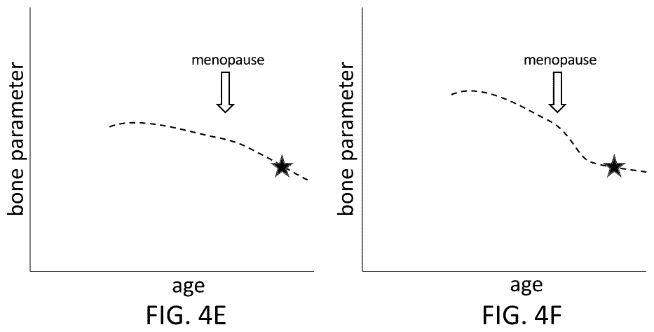

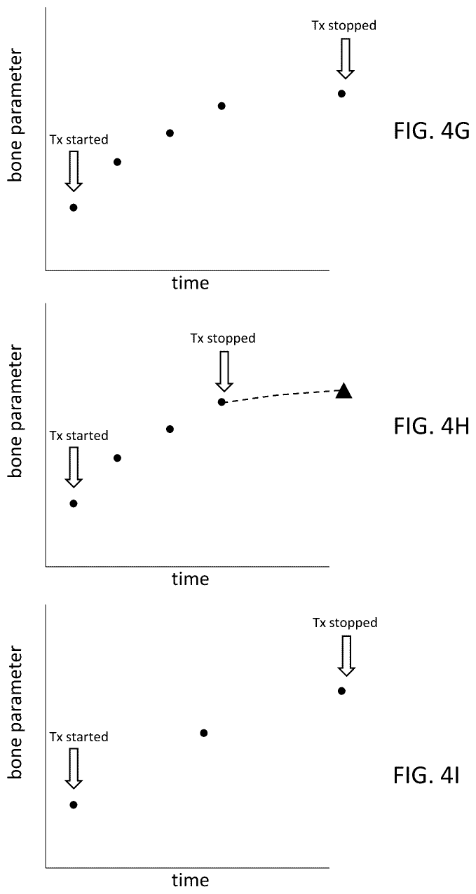

FIG. 4A shows a prior art graph of an example change of a bone parameter with time in a female patient through menopause. In such patients, parameters such as bone density or bone strength are typically highest around the age of 30 and decline thereafter. There is also an increase rate of loss of such parameters after menopause. One of the drawbacks of current clinical management of osteoporosis is that a first time BMD is measured often after menopause i.e., after a significant amount of bone has been lost. The single measurement does not give any indication of whether the patient had a high peak BMD which is rapidly declining or had a low peak BMD that is reducing slowly. In some embodiments of the present disclosure, imaging using one or more methods and/or devices disclosed herein may be performed at one or more of the following time points: around the time of peak bone density and/or strength, before menopause, during menopausal transition, after menopause, at the time of starting a therapy, during a therapy, during the end of a therapy, transitioning between two therapies, etc. One advantage of the methods and devices disclosed herein is that they can be implemented using low cost, portable, easy to use equipment that can be installed in a physician's office (instead of a separate imaging center) thereby improving patient and provider access to such methods and devices without burdening the healthcare system. FIG. 4B shows an embodiment of the present disclosure wherein an imaging step disclosed herein (marked with a star) may be performed after menopause. In this patient, one or more tools (e.g., predictive analytics tools) may be used to determine one or more historical and/or future values of the parameter. FIG. 4C shows the use of the present disclosure to determine one or more historical and/or future values of the parameter (marked with the dashed line) where a low peak bone parameter may be declining slowly. FIG. 4D shows the use of the present disclosure to determine one or more historical and/or future values of the parameter (marked with the dashed line) where a high peak bone parameter may be declining rapidly. FIG. 4E shows the use of the present disclosure to determine one or more historical and/or future values of the parameter (marked with the dashed line) where a low peak bone parameter is declining rapidly. FIG. 4F shows the use of the present disclosure to determine one or more historical and/or future values of the parameter (marked with the dashed line) where a high peak bone parameter is declining slowly. Such predictions may be used, for example, to predict a rapid fall in bone strength and a consequent rapid increase in fracture risk. In such patients, more effective medications (e.g., osteoanabolics) may be administered as a first line therapy to rapidly reduce the fracture risk instead of administering bisphosphonates, which are often the first drugs to be administered.

FIG. 4G shows an embodiment of the present invention wherein multiple measurements or one or more bone parameters are used to stop a treatment. Examples of bone parameters are disclosed elsewhere herein. They include, but are not limited to: measurements of bone strength, BMD (areal, volumetric, etc.), trabecular bone score, etc. In this embodiment, a scan may be performed around the time of starting the treatment. In this embodiment, the treatment may be stopped because peak efficacy has been reached or the bone parameter has been raised to a sufficient level. In one such embodiment, the present disclosure may be used to determine an individualized duration of osteoanabolic therapies.

FIG. 4H shows another embodiment of the present invention wherein multiple measurements or one or more bone parameters may be used to stop a treatment. In this embodiment, a scan may be performed around the time of starting the treatment. In this embodiment, the treatment may be stopped because of one or more of: projected increase in the bone parameter (dashed line) is small, bone parameter has reached close to the projected peak (triangle), etc.

FIG. 4I shows another embodiment of the present invention wherein multiple measurements or one or more bone parameters are used to stop a treatment. In this embodiment, a scan may be performed around the time of starting the treatment. In this embodiment, the bone parameter may be shows a continuous increase. However, the treatment may be stopped because of one or more of: adverse event(s), other inputs such as biochemical measurements, etc.

Any of the methods steps disclosed herein (e.g., the method steps disclosed in FIGS. 2A-4I) may be combined and used for applications including, but not limited to:
1. Selecting type and/or dosage of the drugs, supplements, and/or other therapies
2. Customizing or adjusting the type and/or dosage of therapies by detecting and/or analyzing at the response. One advantage of this disclosure is that the response can be determined at shorter intervals than by existing prior art methods which often show response after more than two years. One of the advantages of the present disclosure is that digital processing techniques and/or imaging methods are used to image and define microstructures and image/calculate even small changes to microstructures in short (e.g., less than about 3 months, less than about 2 months, less than about 1 month, etc.) treatment durations.
3. Determining prognosis by looking at natural progression of bone loss or treatment (e.g., steroid treatment) related bone loss
4. Diagnosing osteoporosis or osteopenia
5. Predicting fracture risk
6. Predicting fracture location
7. Determining ideal drug, exercise, prescription, or other digital therapy, etc. and combinations thereof.
8. Calculating change in one or more bone parameters e.g., bone loss, BMD reduction, change in bone strength from the peak condition which is usually in early adulthood, etc.
9. Improving patients' adherence to drug or other therapies by demonstrating pictorial or quantifiable improvement in one or more bone parameters
10. Using the imaging data to determine the characteristics, type, extent, severity, etc. of osteoporosis. Delivering therapies and/or drugs based on the type of osteoporosis that is found
11. Determining the response of therapies in clinical studies

In any of the embodiments herein (including the embodiments shown in FIGS. 4G-4I), the therapy is romosozumab and the time period of stopping therapy is less than about 1 year (e.g., less than about 10 months, less than about 7 months, etc.). Romosozumab therapy may be stopped within a year after one or more parameters have been met (e.g., sufficient bone has been formed, sufficient bone strength has been gained, osteoanabolic effect is tapering, etc.) to reduce cardiovascular and other risks.

In any of the embodiments herein (including the embodiments shown in FIGS. 4G-4I), the therapy is romosozumab and the time period of stopping therapy is more than about 1 year (e.g., more than about 14 months, more than about 16 months, etc.)

In any of the embodiments herein (including the embodiments shown in FIGS. 4G-4I), the therapy is teriparatide and the time period of stopping therapy is less than about 2 years (E.g., less than about 20 months, less than about 18 months, etc.)

In any of the embodiments herein (including the embodiments shown in FIGS. 4G-4I), the therapy is abaloparatide and the time period of stopping therapy is less than about 2 Years (e.g., less than about 20 months, less than about 18 months, etc.)

In some embodiments, a first drug is administered for some time and the treatment effect measured or analyzed using one or more embodiment disclosed herein. A second drug may be administered for some time and the treatment effect measured or analyzed using one or more embodiments disclosed herein. Thereafter, a determination may be made on the eventual drug therapy by comparing the treatment effect between the two drugs.

Any of the imaging methods disclosed herein may be performed with application of one or more stresses to the imaged bone.

Any of the methods and devices disclosed herein may be used to diagnose and/or monitor and/or treat bone loss and/or bone strength loss in patients experiencing bone loss. Such bone loss may occur in those patients from one or more of: taking corticosteroids or other medications that can cause bone loss, getting a condition that can cause bone loss, etc.

In some embodiments, the present disclosure is used to determine the dosing frequency of injectable medications such as zoledronate or denosumab.

Any of the systems or methods described herein may be used to determine: one or more of: sites, amount, change, etc. of one or more of: bone modeling, bone remodeling, bone growth, and bone loss.

In some embodiments, one of the scans is performed at or near peak bone density. A subsequent scan is performed around menopause.

Any of the scans disclosed herein may be performed before the patient reaches peak bone density. Methods and devices disclosed herein may be used to predict peak bone density or other bone strength parameter. If the predicted bone strength parameter is not sufficient, one or more interventions, (including, but not limited to the interventions disclosed herein) may be performed to increase the peak bone strength or density. Examples of such interventions include, but are not limited to: supplements (like vitamin D, calcium), drugs, exercises, etc. A subsequent scan may be performed to check the effects of such interventions before the patient reaches peak bone strength.

Any of the methods and devices disclosed herein may be used for managing, treating, and/or identifying:
1. any skeletal condition such as osteoporosis, fractures, hypophosphatasia, ankylosing spondylitis, systemic sclerosis, rheumatoid arthritis, etc.
2. skeletal and other effects of procedures like joint replacement,
3. skeletal effects of therapies such as osteoporosis therapies, cancer therapies, corticosteroids, and other.

Any of the methods and devices disclosed herein may be used to manage Osteoclast Rebounding Post Suppression (ORPS). ORPS is defined as the rebound increase in activity of osteoclasts after removing the action of osteoclast suppressing drugs, that is especially seen after stopping the more potent anti-resorptives like denosumab. ORPS may be detected or measured e.g., using the devices and methods described herein. A sufficient amount of anti-resorptive drug(s) may be given to continue osteoclast suppression and prevent ORPS. The amount, duration, type, etc. of the anti-resorptive drug(s) may be determined using one or more embodiments disclosed herein. The anti-resorptive drug(s) may be combined with one or more osteoanabolic drugs to achieve a greater increase in bone strength. In one example, anti-resorptive therapy is followed by osteoanabolic therapy (e.g. with PTH based drug(s)) along with sufficient anti-resorptive therapy to prevent ORPS. Thereafter, the osteoanabolic therapy may be stopped and anti-resorptive therapy may be continued. In another example, a combination of osteoanabolic and anti-resorptive therapy may be administered to a patient. Thereafter, the osteoanabolic therapy may be stopped and only anti-resorptive therapy may be administered. The osteoanabolic therapy may be tapered off during this transition. The first-line drug therapy in many patients is bisphosphonate therapy. However, it doesn't work sufficiently well in many patients. The devices and methods described herein may be used to determine the lack of efficacy of bisphosphonate therapy and manage ORPS post-bisphosphonate therapy. In some embodiments, a follow-on therapy after a duration of anti-resorptive therapy may be either romosozumab or other dual-action drugs or a combination of osteoanabolic and anti-resorptive therapy. One advantage of the devices and methods described herein is that lack of sufficient efficacy of a drug or other therapy may be identified earlier, and a subsequent, more efficacious therapy may be administered earlier than what is currently done.

One example of a therapy sequence includes osteoanabolic therapy followed by combination of osteoanabolic and sufficient anti-resorptive therapy followed by anti-resorptive therapy.

In some embodiments, the degree of ORPS may be predicted by analyzing the amount of osteoclast suppression after anti-resorptive therapy. In one such embodiment, a higher amount of osteoclast suppression may be used to determine the probability and/or degree of ORPS. In some embodiments, the devices and methods described herein may be used for an alternating romosozumab and/or denosumab therapy.

In some embodiments, the devices and methods described herein may be used for an alternating PTH-based osteoanabolic plus anti-resorptive and/or denosumab combination therapy.

In some embodiments, a second scan may be performed at least one month after a first scan. The changes between first and second scan are used to determine a therapy to be administered to the patient. For example, a first scan may be performed before menopause and a second scan may be performed after menopause. The detected changes may be used to treat post-menopausal osteoporosis. A first scan may be performed before starting a drug treatment and a second scan may be performed after starting the drug treatment. The changes between the scans may be used to treat bone or skeletal effects of the drug treatment or the clinical indication of the drug treatment.

In some embodiments, at least three scans of a patient may be performed at separate time points wherein the orientation and/or distance of the bodily organ of interest and one or more device components is matched between successive scans. Thereafter, two or more of such scans may be compared or analyzed.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the scanner and/or computing device. The computer-readable medium can be stored on any suitable computer-readable media such as RAMs, ROMs, flash memory, EEPROMs, optical devices (e.g., CD or DVD), hard drives, floppy drives, or any suitable device. In some embodiments, the computer-readable medium is in electronic communication with an imaging device. In some embodiments, the computer-readable medium is a component of an imaging device. The computer-readable medium may store information from current and prior scans. For example, the computer-readable medium may store information about the positioning and/or orientation of prior scans such that subsequent scans can be performed to ensure consistent imaging. The computer-executable component is preferably a general or application-specific processor, but any suitable dedicated hardware or hardware/firmware combination can alternatively or additionally execute the instructions. The computer-executable component 301, an example of which is shown in FIG. 3C, may be designed to allow parallel processing using a processor, for example a Graphics Processing Units (GPUs). Other exemplary processors may be used such as a Field-Programmable Gate Array, an Application-Specific Integrated Circuits, and the like. The system may use a computer-executable component, one or more computer-executable components, or a plurality of computer-executable components on a computing device, one or more computing device, or a plurality of computing devices. The computing devices may be local, remote, a server, in the Cloud, workstation, etc. The one or more computing devices may be networked, in some embodiments.

Any of the method and device embodiments disclosed herein may be loaded on to or otherwise incorporated into a DXA scanner. In such DXA scanners, the orientation of one or more scanner component(s) and the anatomy may be changed using any of the methods and systems disclosed herein. Such modified DXA scanners and other machines can be used to scan the same patient at multiple times in same/similar/consistent orientations.

In any of the method and device embodiments herein, data (e.g. imaging data) from multiple bones may be generated and/or combined and/or processed together to perform one or more methods disclosed herein.

Although several embodiments of the disclosure are disclosed herein, various modifications (e.g., additions, deletions), combinations, etc. may be made to examples and embodiments herein without departing from the intended spirit and scope of the disclosure. Any component, sensor, etc. of one device embodiment may be incorporated into or used for another device embodiment, unless to do so would render the resulting device embodiment unsuitable for this disclosure. Any suitable device disclosed herein may be used to perform one or more method embodiments disclosed herein. If method steps are disclosed in a particular order, the order of steps may be changed unless doing so would render the method embodiment unsuitable for its intended use. A method step described herein may be added to or used to replace a step of another method embodiment described herein.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “image” may include, and is contemplated to include, a plurality of images. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of treating a bone-related disease, comprising:
performing a first scan of one or more bone regions using an imaging device to obtain first imaging data;
receiving clinical information based on the first imaging data being processed using an artificial intelligence-based model; and
initiating a first therapy after performing the first scan, wherein the clinical information is used to determine one or more parameters of the first therapy.

2. The method of claim 1, wherein the one or more parameters of the first therapy are selected from the group consisting of: an initiation of the first therapy, a non-initiation of the first therapy, a type of the first therapy, a dose of the first therapy, a route of administration, a duration of the first therapy, a timepoint of one or more subsequent scans, and a timepoint of one or more subsequent follow-ups.

3. The method of claim 1, wherein the clinical information is selected from the group consisting of: a bone strength, a bone mineral density, one or more outputs generated by dual-energy X-ray absorptiometry scanners, a bone quality, a bone score, a fracture resistance, one or more locations of bone sites at high risk of fracture, a fracture risk, a presence or absence of osteoporosis, a severity of osteoporosis, a predicted or expected progression of osteoporosis, and a predicted response to one or more therapies.

4. The method of claim 3, wherein the clinical information is a predicted response to a bisphosphonate therapy, and wherein the first therapy is a non-bisphosphonate therapy.

5. The method of claim 3, wherein the clinical information is a probability of treatment success with a drug therapy.

6. The method of claim 5, further comprising selecting the first therapy based on a calculated probability of treatment success of the first therapy.

7. The method of claim 1, wherein the first imaging data is obtained after repositioning the one or more bone regions relative to one or more regions of the imaging device.

8. The method of claim 1, further comprising performing a second scan, wherein the second scan is performed with one or both of: a same distance or a same orientation of the one or more bone regions with the imaging device as the first scan.

9. The method of claim 8, further comprising comparing a result of the first scan with a result of the second scan.

10. The method of claim 9, wherein the comparing is used for one or more of: detecting changes in bone, detecting bone growth, detecting bone loss, detecting effect of a therapy, or a combination thereof.

11. The method of claim 8, further comprising modifying the first therapy after performing the second scan.

12. The method of claim 11, wherein the modifying the first therapy comprises one or more of: changing a dose of the first therapy, stopping the first therapy, starting a second therapy, starting a drug holiday, or stopping a drug holiday.

13. The method of claim 1, further comprising administering a second therapy, wherein the first therapy is an anabolic therapy, and the second therapy is an anti-resorptive therapy.

14. The method of claim 1, further comprising determining a lack of efficacy of the first therapy; and administering a second therapy, wherein the first therapy is an anti-resorptive therapy, the second therapy is an anabolic therapy.

15. The method of claim 1, further comprising performing a second scan, wherein the one or more parameters of the first therapy are modified after the second scan.

16. The method of claim 1, further comprising performing a second scan, wherein the second scan is used to determine one or more of: a loss or tapering of an effect of the first therapy, one or more side-effects of the first therapy, an achievement of a peak therapeutic effect, and a lowering of a rate of bone gain.

17. The method of claim 16, further comprising discontinuing the first therapy and initiating a second therapy.

18. The method of claim 17, wherein the first therapy is an anabolic therapy, and the second therapy is an anti-resorptive therapy.

19. A method of treating a bone-related disease, comprising:
performing a first scan of one or more bones using an X-ray imaging device to obtain first X-ray imaging data;
receiving clinical information based on the first X-ray imaging data being processed using an artificial intelligence-based model;
detecting a presence or a severity of osteoporosis using the artificial intelligence-based model; and
initiating a first therapy after performing the first scan, wherein the clinical information is used to determine one or more parameters of the first therapy.

20. A method of treating a bone-related disease, comprising:
performing a first scan of one or more bone regions using an X-ray imaging device to obtain first X-ray imaging data;
receiving first clinical information based on the first X-ray imaging data being processed using an artificial intelligence-based model;
initiating a first therapy after performing the first scan, wherein the first clinical information is used to determine one or more parameters of the first therapy;
performing a second scan of one or more bones using the X-ray imaging device to obtain second X-ray imaging data, wherein the second scan is performed with one or both of: a same distance or a same orientation of the one or more bone regions with the X-ray imaging device as the first scan;
receiving second clinical information based on the second X-ray imaging data being processed using the artificial intelligence-based model; and
initiating a second therapy after performing the second scan, wherein the second clinical information is used to determine one or more parameters of the second therapy.
